(19) United States
(12) Patent Application Publication
(10) Pub. No.: US 2019/0051039 A1
(43) Pub. Date: Feb. 14, 2019

(54) IMAGE PROCESSING APPARATUS, IMAGE PROCESSING METHOD, PROGRAM, AND SURGICAL SYSTEM

(71) Applicant: SONY CORPORATION, Tokyo (JP)

(72) Inventors: Daisuke TSURU, Chiba (JP); Tsuneo HAYASHI, Tokyo (JP); Yasuaki TAKAHASHI, Kanagawa (JP); Koji KASHIMA, Kanagawa (JP); Kenji IKEDA, Kanagawa (JP)

(73) Assignee: SONY CORPORATION, Tokyo (JP)

(21) Appl. No.: 16/078,057

(22) PCT Filed: Feb. 10, 2017
(86) PCT No.: PCT/JP2017/004846

§ 371 Date: Aug. 21, 2018

(30) Foreign Application Priority Data
Feb. 26, 2016 (JP) 2016-035125

(51) Int. Cl. G06T 15/60; G06T 7/00; A61B 1/05; G06T 15/80; G06T 5/50; A61B 1/06

(52) U.S. Cl. CPC G06T 15/60; G06T 7/0012; A61B 1/05; G06T 15/80; G02B 23/243; A61B 1/0661; G06T 2207/20104; G06T 2207/30004; G06T 5/50

(57) ABSTRACT

The present technology relates to an image processing apparatus, an image processing method, a program, and a surgical system, capable of appropriately providing medical image with shadow/shade.

The image processing apparatus determines whether shadow/shade is to be added or suppressed onto a medical image and controls to generate a shadow/shade corrected image on the basis of a determination result. The present technology can be applied to, for example, a surgical system or the like of performing a surgery while viewing a medical image photographed by an endoscope.

### TECHNICAL FIELD

The present technology relates to an image processing apparatus, an image processing method, a program, and a surgical system, and particularly relates to an image processing apparatus, an image processing method, a program, and a surgical system capable of appropriately providing a medical image with shadow/shade, for example.

### BACKGROUND ART

For example, in endoscopic surgery, surgical site is photographed by an endoscope, and surgery is performed with a view of a medical image in which the surgical site appears.

In the case of using an endoscope, illumination light for illuminating a subject is emitted to the surgical site or the surroundings, and the reflected light is received by the camera so as to photograph a medical image. The endoscope has an optical axis of the illumination light (light source) and an optical axis of the camera substantially matched with each other, so as to leave substantially no shadow to the subject appearing in the medical image.

With such a medical image, it is possible to prevent a situation in which the subject such as a surgical site is hidden behind the shadow and becomes less visible.

On the other hand, however, the subject appearing in the medical image with substantially no shadows makes an image with no irregularity effects (a blunt or flat image). This type of image has a problem that it is difficult to grasp the three-dimensional structure of the subject, and difficult to feel anteroposterior effects (sense of distance in anteroposterior direction) between the subjects (for example, built-in tools, treatment tools such as forceps, and the like) that can be obtained by the manner how shadows are formed, for example.

Note that there is a proposed technique of emphasizing the shadow/shade of a three dimensional (3D) image (for example, Patent Document 1) and a proposed technique of adding shadows to a subject with illumination light applied in a direction orthogonal to the direction of the observation field of the endoscope (for example, Patent Document 2).

### CITATION LIST

### Patent Document

Patent Document 1: Japanese Patent Application Laid-Open No. 2014-022867
Patent Document 2: Japanese Patent Application Laid-Open No. 10-165357

### SUMMARY OF THE INVENTION

### Problems to be Solved by the Invention

An exemplary method of giving irregularity effects, stereoscopic effects, and anteroposterior effects to a medical image is adding shadow/shade to the medical image.

The technique of Patent Document 1, however, is a technique of emphasizing the shadow/shade already existing in a 3D image, and thus, it is sometimes difficult to add shadow/shade to a medical image with substantially no shadow/shade.

Moreover, with the technique of Patent Document 2, a surgical site or the like is indirectly illuminated from sideway by reflected light generated by reflection of illumination light emitted in the direction orthogonal to the direction of the observation field of the endoscope at a wall surface in the body cavity, so as to add shadow/shade to the images photographed by the endoscope.

Therefore, in a case where the surgical site exists over a wide space, reflected light might diffuse, making it difficult to add shadow/shade. Furthermore, it is difficult to add desired shadow/shade.

Meanwhile, successful addition of shadow/shade to a medical image would be able to give the irregularity effects, stereoscopic effects, and anteroposterior effects to the surgeon or the like as a viewer of the medical image.

However, even when it is possible to add shadow/shade to the medical image, there is a case where the surgical site is hidden behind the shadow and becomes less visible, for example.

Therefore, it is not always appropriate to add shadow/shade to medical images.

The present technology has been made in view of such a situation and aims to be able to appropriately display a medical image with shadow/shade.

### Solutions to Problems

An image processing apparatus or program according to the present technology is an image processing apparatus including a control unit that determines whether to add or suppress shadow/shade to a medical image and controls to generate a shadow/shade corrected image on the basis of a result of the determination, or a program that causes a computer to function as an image processing apparatus like this.

An image processing method according to the present technology is an image processing method including steps of determining whether to add or suppress shadow/shade to a medical image and controlling to generate a shadow/shade corrected image on the basis of a result of the determination.

A surgical system according to the present technology includes: an endoscope that photographs a medical image; a light source that emits illumination light for illuminating a subject; and an image processing apparatus that performs image processing on the medical image of the subject illuminated by the illumination light, obtained by photographing with the endoscope, in which the image processing apparatus includes a control unit that determines whether to add or suppress shadow/shade to a medical image and that controls to generate a shadow/shade corrected image on the basis of a result of the determination.

With the image processing apparatus, the image processing method, the program, and the surgical system of the present technology, determination of whether to add or suppress shadow/shade to a medical image is performed and control of generating a shadow/shade corrected image is performed on the basis of a result of the determination.

Note that the image processing apparatus may be a separate apparatus or may be an internal block included in one apparatus.

Moreover, the program can be provided by transmission via a transmission medium, or by being recorded on a recording medium.

### Effects of the Invention

According to the present technology, it is possible to appropriately provide a medical image having shadow/shade, for example.

Note that effects described herein are non-restricting. The effects may be any effects described in the present disclosure.

### BRIEF DESCRIPTION OF DRAWINGS

FIG. 1 is a diagram illustrating shadows and shading.

FIG. 2 is a block diagram Illustrating an exemplary configuration of an endoscope system according to an embodiment of the present technology.

FIG. 3 is a diagram illustrating an example of use of an endoscope system.

FIG. 4 is a diagram illustrating an example of a medical image photographed by an endoscope 11.

FIG. 5 is a block diagram illustrating a first exemplary configuration of an image processing apparatus 12.

FIG. 6 is a diagram illustrating an example of generation of shadow information by a shadow image generation unit 53.

FIG. 7 is a diagram illustrating artifacts occurring in an output image by performing shadow/shade combining processing in a shadow/shade combining processing unit 42.

FIG. 8 is a diagram illustrating artifacts occurring in an output image by performing shadow/shade combining processing in a shadow/shade combining processing unit 42.

FIG. 9 is a flowchart illustrating exemplary processing of the image processing apparatus 12.

FIG. 10 is a block diagram illustrating a second exemplary configuration of the image processing apparatus 12.

FIG. 11 is a flowchart illustrating exemplary processing of the image processing apparatus 12.

FIG. 12 is a diagram illustrating an exemplary output image obtained by the image processing apparatus 12.

FIG. 13 is a block diagram illustrating a third exemplary configuration of the image processing apparatus 12.

FIG. 14 is a diagram illustrating an example of control of a light source 21 in an illumination control unit 71.

FIG. 15 is a diagram illustrating a first example of generation of an output image from a frame of an input image photographed under each of a plurality of (setting) illumination conditions.

FIG. 16 is a diagram illustrating a second example of generation of an output image from a frame of an input image photographed under each of a plurality of illumination conditions.

FIG. 17 is a diagram illustrating a third example of generation of an output image from a frame of an input image photographed under each of a plurality of illumination conditions.

FIG. 18 is a diagram illustrating exemplary processing of the shadow/shade region detection unit 82.

FIG. 19 is a diagram illustrating exemplary processing of a hidden image generation unit 83 and a shadow image generation unit 85.

FIG. 20 is a diagram illustrating exemplary processing of a shadow removing unit 84.

FIG. 21 is a diagram illustrating exemplary processing of the shadow image generation unit 85.

FIG. 22 is a diagram illustrating exemplary processing of a combining unit 86.

FIG. 23 is a diagram illustrating a virtual light source position set by a virtual light source position setting unit 51.

FIG. 24 is a flowchart illustrating exemplary processing of the image processing apparatus 12.

FIG. 25 is a block diagram illustrating a fourth exemplary configuration of the image processing apparatus 12.

FIG. 26 is a block diagram illustrating a fifth exemplary configuration of the image processing apparatus 12.

FIG. 27 is a diagram illustrating exemplary shade region detection by a shade region detection unit 111.

FIG. 28 is a flowchart illustrating exemplary processing of the image processing apparatus 12.

FIG. 29 is a block diagram illustrating an exemplary configuration of a computer according to an embodiment of the present technology.

### MODE FOR CARRYING OUT THE INVENTION

<Shading and Shadows>

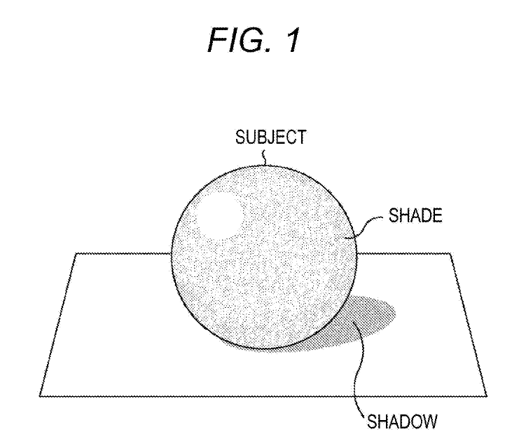

FIG. 1 is a diagram illustrating shadows and shade.

In FIG. 1, illumination light is emitted from the front side on the diagonally upper left toward a subject. As illustrated in FIG. 1, a shade means a dark portion not irradiated with light (illumination light), and a shadow means a dark portion formed by shielding the light by an object (subject).

In the present specification, the term “shadow/shade” represents a shade alone, a shadow alone, or both shade and shadow.

<One embodiment of endoscope system according to the present technology>

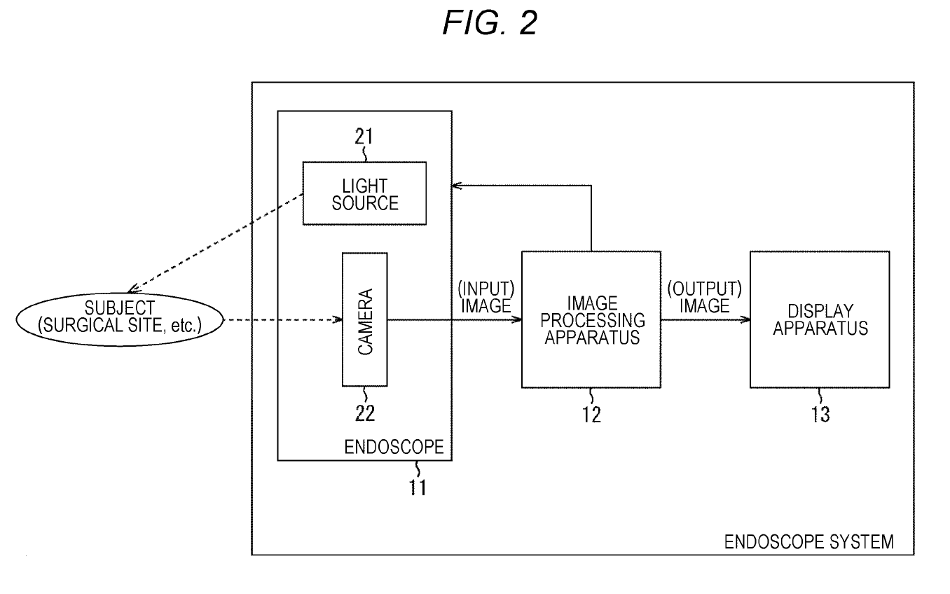

FIG. 2 is a block diagram illustrating an exemplary configuration of an endoscope system according to an embodiment of the present technology.

In FIG. 2, the endoscope system includes an endoscope 11, an image processing apparatus 12, and a display apparatus 13.

For example, the endoscope 11 photographs a subject being a living body such as a surgical site of a human body to be treated under illumination, and supplies a medical image of the surgical site obtained by the photographing as an input image to the image processing apparatus 12 as an input image to the image processing apparatus 12.

The endoscope 11 is an imaging unit that includes a light source 21 and a camera 22, and uses the camera 22 to photograph a subject such as a surgical site illuminated by the light source 21.

The light source 21 includes, for example, a light emitting diode (LED) and the like and emits illumination light for illuminating a subject such as a surgical site.

The camera 22 includes, for example, an optical system, an image sensor (neither is illustrated) such as a complementary metal oxide semiconductor (CMOS) sensor, and the like. The camera 22 receives subject light (reflected light) incident by reflection of the illumination light emitted from the light source 21 being reflected by the subject so as to photograph a medical image of a subject such as a surgical site and supply the medical image to the image processing apparatus 12 as an input image.

Note that the camera 22 is capable of photographing, as a medical image, a two-dimensional (2D) image and a three-dimensional (3D) image including an image for the left eye (left (L) image) and an image for the right eye (right (R) image).

The image processing apparatus 12 performs a shadow/shade processing described below and other necessary image processing on a medical image from the endoscope 11 (specifically, the camera 22), and supplies an image obtained as a result of the image processing to the display apparatus 13 as an output image to the display apparatus 13.

In addition, the image processing apparatus 12 controls the endoscope 11 as necessary.

That is, for example, the image processing apparatus 12 controls the light source 21 so as to control illumination light emitted from the light source 21. In addition, the image processing apparatus 12 controls the camera 22 to adjust a diaphragm, focusing (positions), and zooming, for example. Furthermore, the image processing apparatus 12 controls the camera 22 to control frame rates of a medical image and the exposure time (shutter speed) in photographing the medical image, for example.

The display apparatus 13 displays the image supplied from the image processing apparatus 12. Examples of the adoptable display apparatus 13 include a display integrated with the image processing apparatus 12, a stationary display separate from the image processing apparatus 12, a head mounted display, and the like.

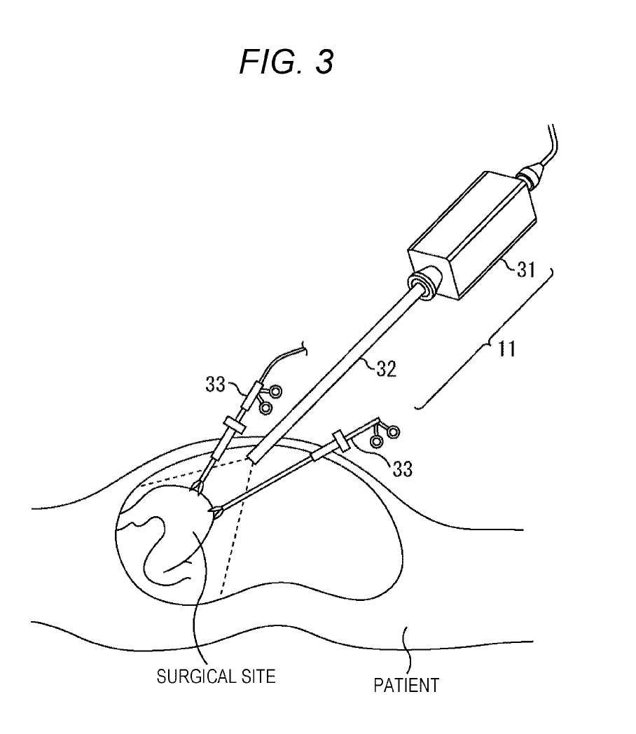

FIG. 3 is a diagram illustrating an example of use of the endoscope system of FIG. 2.

The endoscope system illustrated in FIG. 2 photographs a surgical site (affected site), which is an internal body site as a surgical target as a subject, and displays an endoscopic image being a medical image of the subject, on the display apparatus 13, for example. The endoscope system illustrated in FIG. 2 is used in an endoscopic surgery or the like performed by a doctor as a surgeon to apply treatment on the surgical site while viewing the medical image (endoscopic image).

The endoscope 11 is inserted into a body cavity of a patient (human body) to photograph a medical image having a surgical site in the body cavity as a subject, for example.

Specifically, the endoscope 11 includes, as an external view, for example a camera head 31 manually operated by a surgeon (doctor) or the like performing surgery as a user of the endoscope system, an elongated tubular endoscope scope 32 to be inserted into the body of the patient, and the like.

In endoscopic surgery, the endoscope scope 32 of the endoscope 11 and a treatment tool are inserted into the body of the patient as illustrated in FIG. 3, for example. Here, among treatment tools such as an energy device and forceps, FIG. 3 illustrates a case where forceps 33 are inserted in the patient's body.

With the endoscope 11, illumination light emitted from the light source 21 is applied from the distal end of the endoscope scope 32, and the surgical site as the subject inside the patient's body is illuminated by the illumination light, for example. Furthermore, with the endoscope 11, the reflected light of the illumination light reflected by the surgical site is incident from the distal end of the endoscope scope 32 and received by the camera 22 built in the camera head 31, whereby the surgical site as a subject is photographed.

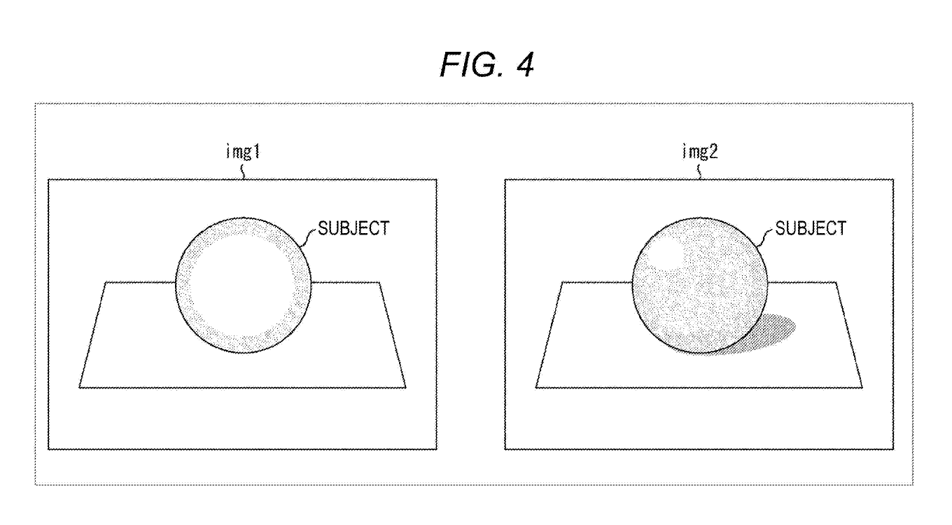

FIG. 4 is a schematic diagram illustrating an example of a medical image photographed by the endoscope 11 of FIG. 2.

The endoscope 11 allows an optical axis of the illumination light emitted from the light source 21 to substantially match with the optical axis of the camera 22. Therefore, almost no shadow is generated in the subject appearing in the medical image appearing by the camera 22.

With such a medical image, it is possible to prevent a situation in which the subject such as a surgical site is hidden behind the shadow and becomes less visible.

The medical image with substantially no shadows, however, makes an image with no irregularity effects (a flat image) like an image img1. This type of medical image has a problem that it is difficult to grasp the three-dimensional structure of the subject, and difficult to feel anteroposterior effects (sense of distance in anteroposterior direction) between the subjects (for example, built-in tools, treatment tools such as forceps, and the like) that can be obtained by the manner how shadows are formed, for example.

To cope with this, the endoscope system illustrated in FIG. 2 has a configuration in which the image processing apparatus 12 sets a virtual light source and performs shadow/shade processing of adding or suppressing shadow/shade on the medical image photographed by the camera 22 so as to adjust the shadow/shade of the image.

Specifically, the image processing apparatus 12 sets a virtual light source at a position diagonally 45 degrees direction with respect to the optical axis of the camera 22, for example, and the shadow/shade processing corresponding to the virtual light source is applied on the medical image, for example. With this processing, the image processing apparatus 12 generates a medical image that appears to be the surgical site as the subject being illuminated by the illumination light emitted from the virtual light source.

The medical image that has undergone the shadow/shade processing of the image processing apparatus 12 is an image having irregularity effects, stereoscopic effects, and anteroposterior effects (distance effects between two objects) as illustrated in an image img2.

With the medical image that has undergone such shadow/shade processing, the surgeon can easily grasp a surface structure (shape) and a spatial position of the subject appearing in the medical image and a positional relationship between the subjects (objects), or the like, leading to achievement of smooth surgery.

Here, a medical image in a 3D image has more stereoscopic effects or the like as compared with the 2D image. Still, the 3D image as the medical image photographed by the endoscope 11 has small parallax, sometimes making it difficult to grasp the position in the depth direction even with the 3D image.

In contrast, with the medical image such as the image img2 that has undergone the shadow/shade processing, it is possible to easily grasp the position in the depth direction.

On the other hand, the medical image that has undergone the shadow/shade processing has another problem that the surgical site appearing in the medical image might be hidden behind the shadow, causing less visibility.

Therefore, it is not always appropriate to perform shadow/shade processing on medical images.

Accordingly, in the endoscope system illustrated in FIG. 2, the image processing apparatus 12 performs shadow/shade necessity determination as to whether shadow/shade processing is to be performed on a medical image, and shadow/shade processing is performed in accordance with the judgment result of the shadow/shade necessity judgment so as to appropriately provide a medical image having shadow/shade.

<First Exemplary Configuration of Image Processing Apparatus 12>

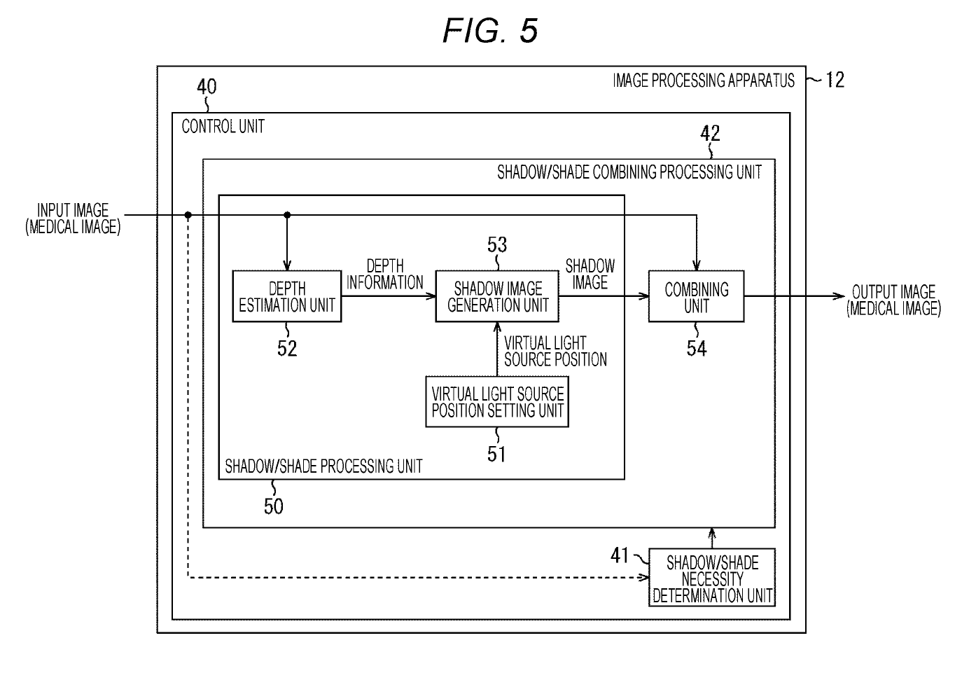

FIG. 5 is a block diagram illustrating a first exemplary configuration of an image processing apparatus 12 in FIG. 2.

In FIG. 5, the image processing apparatus 12 includes a control unit 40.

The control unit 40 includes a shadow/shade necessity determination unit 41 and a shadow/shade combining processing unit 42, and performs various types of control. That is, the control unit 40 performs shadow/shade necessity determination of determining whether to add or suppress shadow/shade to a medical image as an input image from the camera 22, and performs control of generating a shadow/shade corrected image being an image obtained by performing correction related to shadow/shade on the input image, of the like, on the basis of the result of the shadow/shade necessity determination, for example.

The shadow/shade combining processing unit 42 includes a shadow/shade processing unit 50 and a combining unit 54.

The shadow/shade processing unit 50 includes a virtual light source position setting unit 51, a depth estimation unit 52, and a shadow image generation unit 53.

The shadow/shade necessity determination unit 41 performs shadow/shade necessity determination as to whether shadow/shade processing for adding or suppressing shadow/shade is to be performed on a medical image as an input image from the camera 22 and controls (processing of) the shadow/shade combining processing unit 42 on the basis of a shadow/shade necessity determination result (result of the shadow/shade necessity determination).

Here, the shadow/shade necessity determination unit 41 can perform the shadow/shade necessity determination, for example in accordance with operation of the user such as a surgeon, the medical image, and the use situation of the treatment tool, and the like.

That is, the surgeon can operate the endoscope system so as to perform shadow/shade processing to add shadow/shade in a case where it is desired to observe irregularities, shapes, contours, or the like, of a specific subject such as a surgical site, for example. In this case, in the shadow/shade necessity determination unit 41 determines that shadow/shade processing of adding shadow/shade is performed in accordance with the operation by the user.

Furthermore, for example, in a case where laparoscopy and endoscopy cooperative surgery (LECS) using a plurality of endoscopes (laparoscopes) is performed, the surgeon can operate the endoscope system so as to perform the shadow/shade processing by adding shadow/shade as necessary.

In this case, when it is determined in the shadow/shade necessity determination that the shadow/shade processing for adding the shadow/shade is to be performed in accordance with user's operation and then when the shadow/shade processing is actually performed, for example, the surgeon can grasp the position of the endoscope from the shadow of the endoscope operated by another surgeon or the like, appearing on the medical image after shadow/shade processing. In addition, the surgeon can grasp the position and orientation of the endoscope, for example, from the shadow of the endoscope operated by the surgeon. Furthermore, for example, even when the treatment tool is not in the field of view, in a case where the shadow of the treatment tool that is not in the field of view appears in the medical image after the shadow/shade processing, the surgeon can grasp the position of the treatment tool outside the field of view, by the shadow.

In addition, the shadow/shade necessity determination can be performed depending on whether the surgeon wishes to grasp the depth or the anteroposterior relationship.

Examples of the case where the surgeon wishes to grasp the depth and the anteroposterior relationship include cases where suturing is performed, or treatment is performed by a stapler, an energy device, or the like.

The shadow/shade necessity determination unit 41 can recognize, for example, that suturing is being performed, and that the treatment is being performed with a stapler, an energy device, or the like, by detecting a scene appearing in a medical image, for example. Then, in a case where suturing is being performed (for example, in a case where a needle or thread appears in the medical image), or where a treatment is being performed with a stapler, an energy device or the like (a stapler or an energy device appear in the medical image, for example), the shadow/shade necessity determination unit 41 can determine that the shadow/shade processing of adding shadow/shade is to be performed.

Furthermore, the shadow/shade necessity determination unit 41 can recognize that treatment is being performed with an energy device or the like from a use situation of the energy device or the like, that is, an on/off state of a switch or the like. Then, in a case where the treatment is being performed with an energy device or the like, the shadow/shade necessity determination unit 41 can determine that the shadow/shade processing for adding the shadow is to be performed.

As described above, after the shadow/shade necessity determination is performed, with execution of the shadow/shade processing of adding the shadow/shade to the medical image in a case where the treatment is performed with the stapler, the energy device, or the like, the surgeon can easily grasp the distance to the target for treatment with the stapler, the energy device, or the like, for example. Moreover, in a case of pinching a tissue with a stapler, pinching the tissue at an appropriate depth, that is, not too shallow or not too deep. In order to pinch the tissue with an appropriate depth with the stapler, it is necessary to accurately grasp the thickness of the tissue. In this respect, with the medical image that has undergone the shadow/shade processing of adding a shadow/shade, the surgeon can grasp the thickness of the tissue accurately.

Furthermore, the shadow/shade necessity determination can be performed in accordance with the luminance of the medical image.

That is, the shadow/shade necessity determination unit 41 recognizes a surgical site appearing in a medical image, and in a case where the luminance of at least a portion of the surgical site is significantly lower than the surrounding luminance, it is possible to determine that the shadow/shade processing of suppressing the shadow, that is, a portion having low luminance is going to be performed. In this case, the shadow overlapping the surgical site is suppressed in the medical image, making it possible to prevent the case where the surgical site is hidden behind the shadow and becomes less visible.

A medical image photographed by the camera 22 is supplied to the shadow/shade combining processing unit 42 as an input image.

Under the control of the shadow/shade necessity determination unit 41, the shadow/shade combining processing unit 42 performs shadow/shade combining processing on the input image from the camera 22. Thereafter, the shadow/shade combining processing unit 42 supplies the medical image that has undergone the shadow/shade combining processing as an output image to the display apparatus 13, or supplies the input image from the camera 22 to the display apparatus 13 as an output image as it is without applying the shadow/shade combining processing (shadow/shade processing and combining processing described below).

Here, the shadows/shade combining processing performed by the shadow/shade combining processing unit 42 includes shadow/shade processing performed by the shadow/shade processing unit 50 and combining processing performed by the combining unit 54.

Here, the combining processing is performed by using the shadow image or the like in which shadows appear, obtained by the shadow/shade processing. Accordingly, in a case where it is determined that the shadow/shade processing is not to be performed by the shadow/shade necessity determination, neither the shadow/shade processing nor the combining processing is to be performed. In this sense, the shadow/shade necessity determination is determination of necessity of not only the shadow/shade processing, but also the combining processing or shadow/shade combining processing (shadow/shade processing and combining processing).

The shadow/shade (combining) processing includes processing of adding shadow/shade to an input image (medical image) and processing of suppressing shadow/shade occurring in the input image.

Addition of shadow/shade includes not only addition of shadow/shade to portions with no shadow/shade, but also includes addition of darker shadow/shade to the portion with shadow/shade, enlarging (expanding) the range of portion with shadow/shade, for example that is, emphasis of shadow/shade.

In addition, suppression of shadow/shade includes complete suppression of shadow/shade, that is, removal of shadow/shade, as well as reduction of the shade density and reduction of the range of portions with shadow/shade.

In the combining processing, a shadow image or the like generated by the shadow/shade processing is combined into an input image or the like, so as to generate a combined image in which a shadow is added to the subject appearing in the input image, or a combined image from which the shadow of the subject appearing in the input image is removed, as an output image, for example.

The virtual light source position setting unit 51 sets the position of the virtual light source according to the user's operation, for example, and supplies the set position to the shadow image generation unit 53.

For example, when the user performs an operation to specify a direction to which a shadow is to be added, the virtual light source position setting unit 51 sets the virtual light source position at a position opposite to the direction in which the shadow is to be added.

Note that the virtual light source position setting unit 51 can also set, for example, a recommended fixed position (for example, at a position in a direction having a diagonal angle of 45 degrees with respect to the optical axis of the camera 22 at an intersection between the optical axis of the camera 22 and the subject) as a default virtual light source position.

In addition, for example, the virtual light source position setting unit 51 can perform scene detection of the medical image and can set a position where there is no overlapping between the longitudinal direction of the elongated treatment tool such as forceps and the light ray from the virtual light source as the virtual light source position. In this case, it is possible to prevent a case where the shadow/shade processing suppresses generation of the shadow of the treatment tool such as the forceps.

A medical image as an input image is supplied from the camera 22 to the depth estimation unit 52.

Here, the camera 22 photographs a 3D image, for example, and a 3D image is supplied as an input image (medical image) from the camera 22 to the depth estimation unit 52.

Here, the 3D image represents two images (an image for the left eye (L image) and an image for the right eye (R image)) having parallax that enables stereoscopic viewing. The description of “3D image” below follows the above in a similar manner.

The depth estimation unit 52 uses an input image being a 3D image from the camera 22 to estimate the parallax of each of pixels of the input image, and further estimate depth information, that is, information of the distance in the depth direction (direction of the optical axis of the camera 22) of the subject appearing in each of the pixels, and supplies the estimated depth information to the shadow image generation unit 53.

On the basis of the virtual light source position from the virtual light source position setting unit 51 and the depth information from the depth estimation unit 52, the shadow image generation unit 53 generates a shadow image of a shadow generated by the virtual light source (shadow image in which a shadow appears) about the subject appearing in the input image and supplies the image to the combining unit 54.

The shadow image is supplied from the shadow image generation unit 53 to the combining unit 54, and a medical image as an input image is supplied from the camera 22 to the combining unit 54 as well.

The combining unit 54 performs combining processing of combining the input image from the camera 22 and the shadow image (shadow region among the image, to be described below) from the shadow image generation unit 53 so as to generate an output image obtained by adding a shadow to the medical image, and outputs (supplies) the output image to the display apparatus 13.

Note that a shadow image or a combined image obtained by combining a shadow image and an input image can be defined as the above-described shadow/shade corrected image (image obtained by performing correction related to shadow/shade onto the input image).

Meanwhile, combining of the input image and the shadow image in the combining unit 54 can adopt alpha blending, for example.

A coefficient α of alpha blending can be set to a fixed value or can be set to a value according to the user's operation, for example. While the coefficient α is set to have a value range 0.0 to 1.0, with setting of the coefficient α 0.0 or 1.0, it is possible to select no shadow on the input image or to replace the pixel of the input image into the pixel on which the shadow alone appears.

Furthermore, while FIG. 5 is a case where the shadow/shade combining processing unit 42 outputs the combined image obtained by combining the input image and the shadow image in the combining unit 54 as the output image, the combining of the input image and the shadow image can be performed at the time of displaying the input image and the shadow image, rather than by the combining unit 54.

That is, the shadow/shade combining processing unit 42 can output each of the input image and the shadow image as an output image, for example. In this case, for example, it is possible to display a shadow image on a transmissive display apparatus such as a transmissive head mounted display or a glass type wearable device while displaying an input image on the display apparatus 13, so as to provide a combined image obtained by combining the input image and the shadow image. Alternatively, it is possible to arrange a first display panel with transparency on the upper side (the side facing the user) of the second display panel with or without transparency to configure the display apparatus 13, with the shadow image displayed on the first display panel, while displaying the input image on the second display panel, so as to display a combined image obtained by combining the input image and the shadow image.

Moreover, while FIG. 5 illustrates a case where the camera 22 photographs a 3D image as an input image and the depth estimation unit 52 estimates the depth information from the 3D image as an input image, method for estimating the depth information is not limited to a method using 3D images.

That is, for example, the camera 22 can photographs a 2D image as an input image. In this case, it is possible to have a distance sensor (depth sensor) incorporated in the endoscope 11 (FIG. 1), and to enable the depth estimation unit 52 to estimate the depth information on the basis of a 2D image as an input image and a sensing result of the distance sensor.

Here, the 2D image represents one image. The description of “2D image” below follows the above in a similar manner.

In addition, the depth estimation unit 52 can use, for example focus information and the like for estimating the depth.

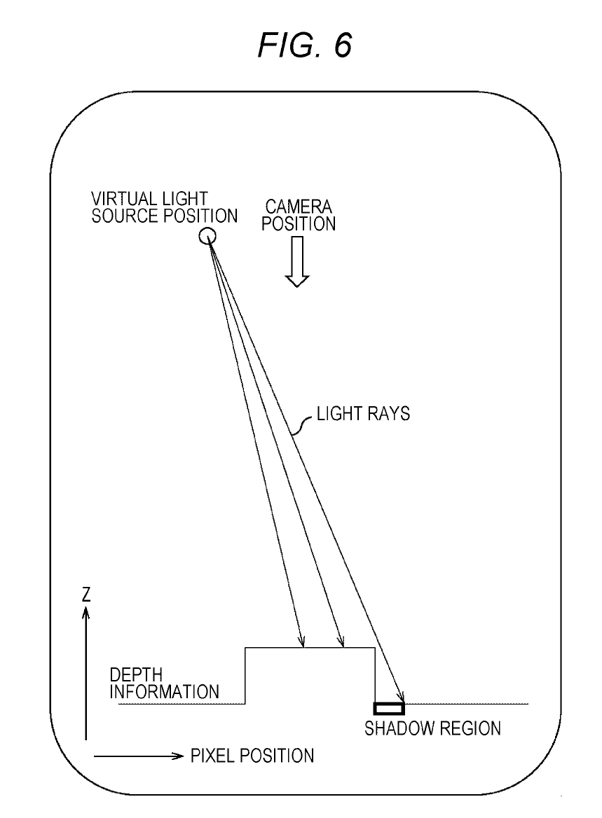

FIG. 6 is a diagram illustrating an example of generation of shadow information by the shadow image generation unit 53 in FIG. 5.

In FIG. 6, the horizontal axis represents the position of each of pixels of the input image, and the vertical axis represents depth information.

The shadow image generation unit 53 obtains (estimates) a region constituted with pixels (of depth information) to which light rays cannot reach as a result of being blocked by depth information of other pixels when light rays (straight lines indicating light rays) are drawn from the virtual light source position toward each of the pixels (the position of the pixels) of the input image, as a shadow region, that is, a region of a shadow generated by the virtual light source.

Furthermore, the shadow image generation unit 53 generates an image in which the pixel value of the pixel of the shadow region is set to a preset color or color set by user's operation, such as black or dark color close to black, as the shadow image, for example.

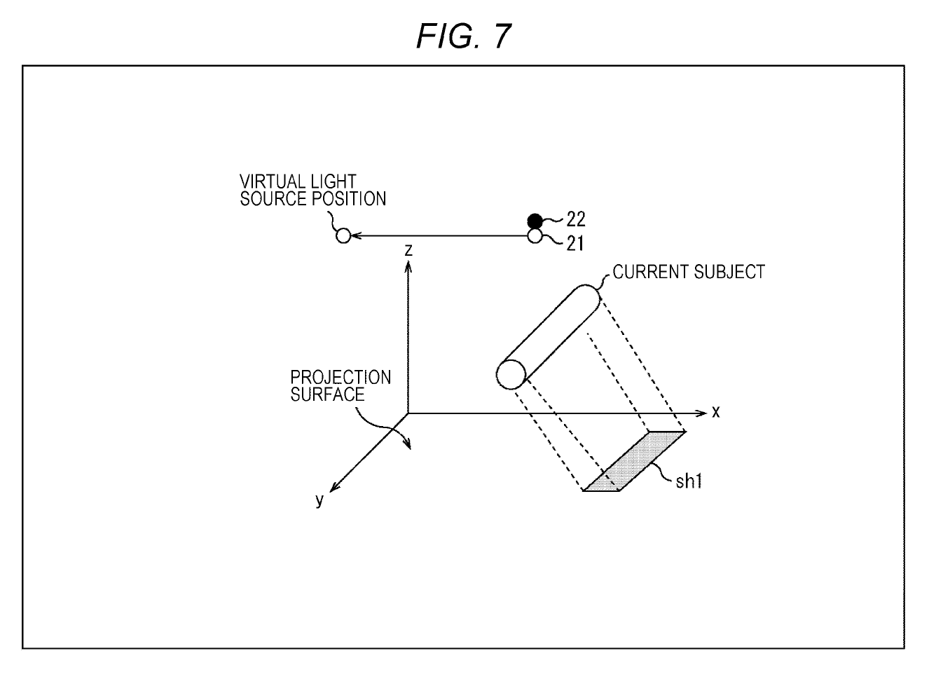

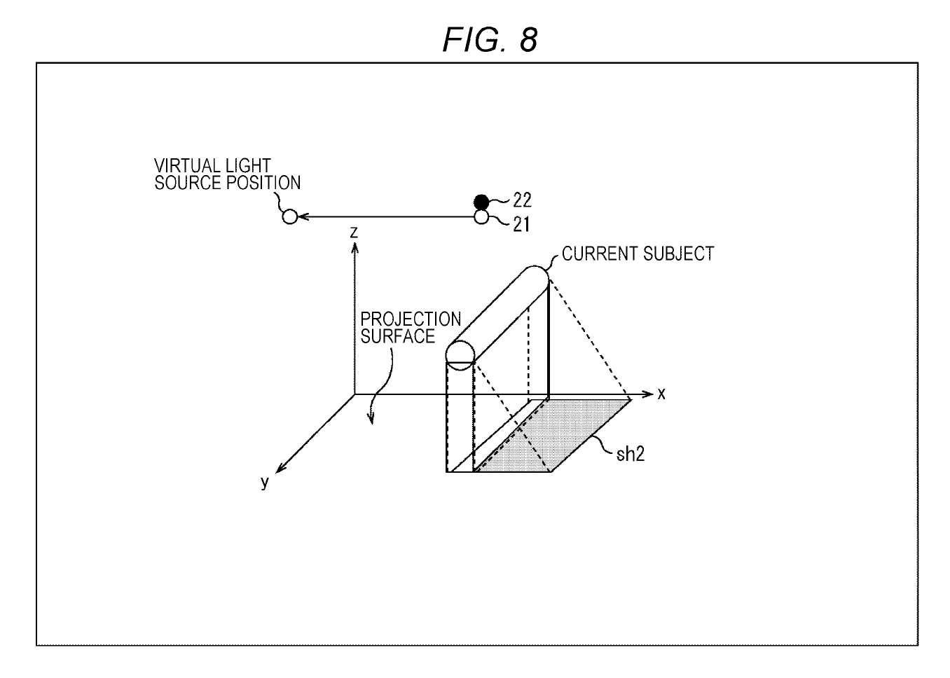

FIGS. 7 and 8 are diagrams each illustrating an artifact occurring in an output image by performing shadow/shade processing by the shadow/shade combining processing unit 42 in FIG. 5.

In the endoscope 11, the light source 21 (position of emitting the illumination light) and the camera 22 can be considered to be located at substantially a same position.

On the assumption that the illumination light is emitted from the virtual light source position set to a position different from the position of the light source 21, the shadow image generation unit 53 obtains the region constituted with the pixels to which light rays as illumination light from the virtual light source position toward each of the pixels of the input image cannot reach as a result of being blocked by depth information of other pixels, as the shadow region as described in FIG. 6.

As described above, the shadow image generation unit 53 obtains the shadow region using the virtual light source position and the depth information. Accordingly, there is a case where a shadow region that is not supposed to arise might arise (appear) in the output image as an artifact depending on the virtual light source position and the position of the subject appearing in the input image.

That is, a subject that currently produces a shadow, that is, a subject that blocks a light ray from the virtual light source position will also be referred to as a target subject.

In the case where the target subject is an elongated treatment tool having a relatively small thickness, such as forceps, an energy device, or the like, a shadow sh1 having an elongated shape is to be generated as illustrated in FIG. 7 while the position of generation of the shadow differs depending on the virtual light source position.

Meanwhile, the shadow image generation unit 53 obtain the shadow region on assumption that the target subject is solid (present) toward the back side (the deeper side) of the target subject as viewed from the camera 22, as illustrated in FIG. 6.

Accordingly, for example, in a case where a virtual light source position is set such that the distance to the optical axis of the camera 22 is large, or in a case where there is a long distance between the position of the target subject blocking the light rays from the virtual light source position and the projection surface on which the shadow of the target subject (shadow generated by the target subject) is projected, the shadow image generation unit 53 obtains a shadow region sh2 that seemingly indicates a state where the target subject is solid up to the projection surface, as illustrated in FIG. 8.

That is, in a case where a virtual light source position is set such that the distance to the optical axis of the camera 22 is large, or in a case where there is a long distance between the position of the target subject blocking the light rays from the virtual light source position and the projection surface on which the shadow of the target subject (shadow generated by the target subject) is projected, the depth information would be a solid protruding model with respect to the projection surface. This makes it difficult to correctly project a shadow of the target subject generated by the illumination light from the virtual light source with the depth information alone.

In a case where a shadow image of such a shadow region sh2 and the input image are combined with each other, the shadow region sh2 not supposed to arise as an artifact arises in the output image obtained by the combining.

To cope with this, the virtual light source position setting unit 51 (FIG. 5) can restrict the distance between the optical axis of the camera 22 and the virtual light source position within a predetermined distance in setting the virtual light source position.

Alternatively, it is possible to restrict addition of a shadow to a target subject when the distance between the position of the target subject and the projection surface on which the shadow of the target subject is projected is a certain distance or more. That is, for example, it is possible to allow the combining unit 54 to suppress combining the shadow region obtained for the target subject having a distance between the position of the target subject and the projection surface onto which the shadow of the target subject is projected is a certain distance or more, with the input image. Moreover, it is possible to allow the shadow image generation unit 53 to suppress generation of the shadow region, or a shadow image itself, for the target subject having a distance between the position of the target subject and the projection surface onto which the shadow of the target subject is projected is a predetermined distance or more.

Alternatively, it is possible to allow the combining unit 54 to adjust the coefficient α in alpha blending of the input image and the shadow image so as to make the artifact as the shadow region that is not supposed to occur less visible.

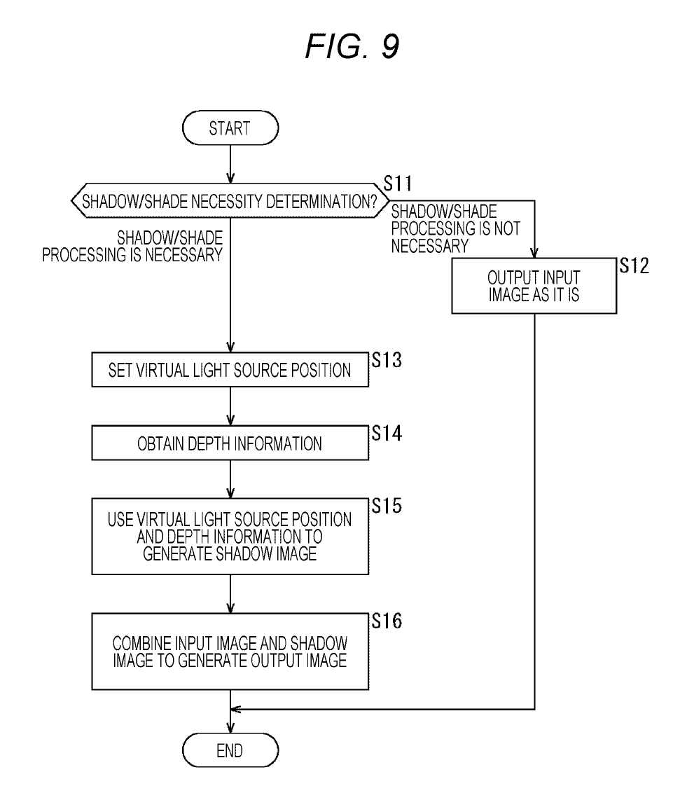

FIG. 9 is a flowchart illustrating exemplary processing of the image processing apparatus 12 in FIG.

In step S11, the shadow/shade necessity determination unit 41 performs shadow/shade necessity determination.

In a case where it is determined in the shadow/shade necessity determination in step S11 that the shadow/shade processing is not necessary for the input image from the camera 22, the processing proceeds to step S12, and then, the shadow/shade combining processing unit 42 outputs the input image from the camera as it is to the display apparatus 13 as an output image, and the processing is finished.

In another case where it is determined in the shadow/shade necessity determination in step S11 that the shadow/shade processing is necessary for the input image from the camera 22, the processing proceeds to step S13, and then, the virtual light source position setting unit 51 sets the virtual light source position and supplies the position to the shadow image generation unit 53. Then, the processing proceeds from step S13 to step S14, and shadow/shade combining processing (shadow/shade processing and combining processing) is performed as below.

That is, in step S14, the depth estimation unit 52 estimates and obtains the depth information from the 3D image as the input image from the camera 22, supplies the information to the shadow image generation unit 53, and the processing proceeds to step S15.

In step S15, the shadow image generation unit 53 generates the shadow image, that is, an image of the shadow generated by the virtual light source as described in FIG. 6, on the basis of the virtual light source position from the virtual light source position setting unit 51 and the depth information from the depth estimation unit 52, and supplies the image to the combining unit 54, and the processing proceeds to step S16.

In step S16, the combining unit 54 combines the input image from the camera 22 and the shadow image (shadow region in the shadow image) from the shadow image generation unit 53 with each other to generate an output image in which a shadow is added to the medical image, and outputs the image to the display apparatus and then, the processing is finished.

Note that while FIG. 5 illustrates a case where the image processing apparatus 12 performs addition of a shadow as shadow/shade processing, the image processing apparatus 12 is also capable of suppressing shadows in addition to addition of shadows. The suppression of shadows can be performed by setting the position of the light source 21 as the virtual light source position to generate a shadow image, and then removing the shadow region of the shadow image from the input image, for example. The portion in the input image from which the shadow region is removed (hereinafter also referred to as a removed portion) can be interpolated by the latest input image among the past input images having no shadows in the removed portion, for example.

<Second Exemplary Configuration of Image Processing Apparatus 12>

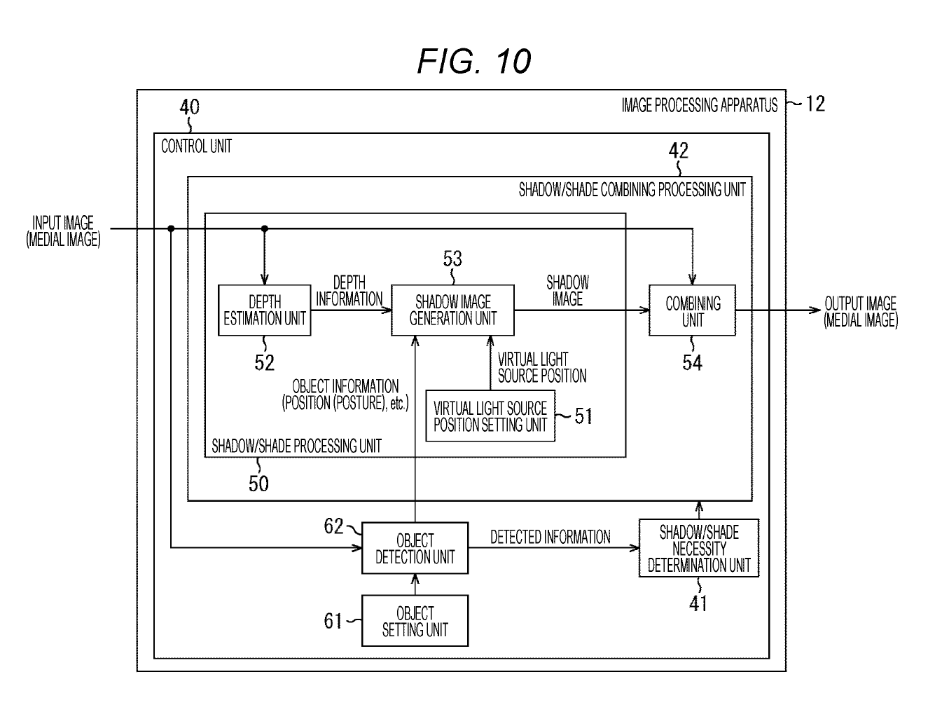

FIG. 10 is a block diagram illustrating a second exemplary configuration of an image processing apparatus 12 in FIG. 2.

Note that in the figure, portions corresponding to the case of FIG. 5 are denoted by the same reference numerals, and the description thereof will be omitted as appropriate below.

In FIG. 10, the image processing apparatus 12 includes the control unit 40.

The control unit 40 includes the shadow/shade necessity determination unit 41, the shadow/shade combining processing unit 42, an object setting unit 61, and an object detection unit 62.

The shadow/shade combining processing unit 42 includes a shadow/shade processing unit 50 and a combining unit 54.

The shadow/shade processing unit 50 includes a virtual light source position setting unit 51, a depth estimation unit 52, and a shadow image generation unit 53.

This allows the image processing apparatus 12 of FIG. 10 to have a similar configuration of FIG. 5 in that it includes the control unit 40, and that the control unit 40 includes the shadow/shade necessity determination unit 41 and the shadow/shade combining processing unit 42.

The image processing apparatus 12 of FIG. 10, however, is different from the case of FIG. 5 in that the object setting unit 61 and the object detection unit 62 are newly provided in the control unit 40.

The object setting unit 61 sets a target object that is an object to be a target of the shadow/shade processing in accordance with, for example, user's operation or the like, and supplies the object information to the object detection unit 62.

Note that the object setting unit 61 can set the target object in accordance with user's operation, and in addition to this, for example can set a predetermined object such as a treatment tool, a needle, and a thread used for surgery, as the target object.

For example, in a case where the user wishes to observe irregularities, shapes, contours of a subject, the user can set an affected site or organ excluding surgical instruments or the like as a target object, for example. In addition, in a case of performing the LECS, the user can set treatment tools including a treatment tool outside the operation field as the target object, for example. Furthermore, for example, in application of suturing, or a treatment using a stapler or an energy device, for example, the user can set a needle, a thread, and a treatment tool as the target object. In addition, the object setting unit 61 can set an object located at a position-of-interest in which the user is interested, or a focus position, as the target object, for example.

An input image (medical image) is supplied from the camera 22 to the object detection unit 62, and together with this, the target object (information indicating the target object) is supplied from the object setting unit 61 to the object detection unit 62.

The object detection unit 62 detects (specifies) the target object from the input image. Then, in a case where the target object can be detected from the input image, the object detection unit 62 generates object information for specifying the target object in the input image, such as the position (region) and posture of the target object in the input image and supplies the information to the shadow image generation unit 53.

Note that the object detection unit 62 can supply detection information indicating whether the target object has been detected from the input image to the shadow/shade necessity determination unit 41.

In this case, the shadow/shade necessity determination unit 41 can perform shadow/shade necessity determination in accordance with the detection information from the object detection unit 62, in addition to description of FIG. 5, or in place of the description of FIG. 5.

That is, the shadow/shade necessity determination can determine to perform shadow/shade processing in a case where the detection information indicates that the target object has been detected, and can determine not to perform shadow/shade processing in a case where the detection information indicates that the target object has not been detected.

In FIG. 10, the shadow image generation unit 53 sets solely the target object specified by the object information from the object detection unit 62 among the subjects appearing in the input image as a target for generation, and generates a shadow image, that is, an image of a shadow of the target object, generated by the virtual light source on the basis of the virtual light source position and the depth information, and supplies the generated image to the combining unit 54.

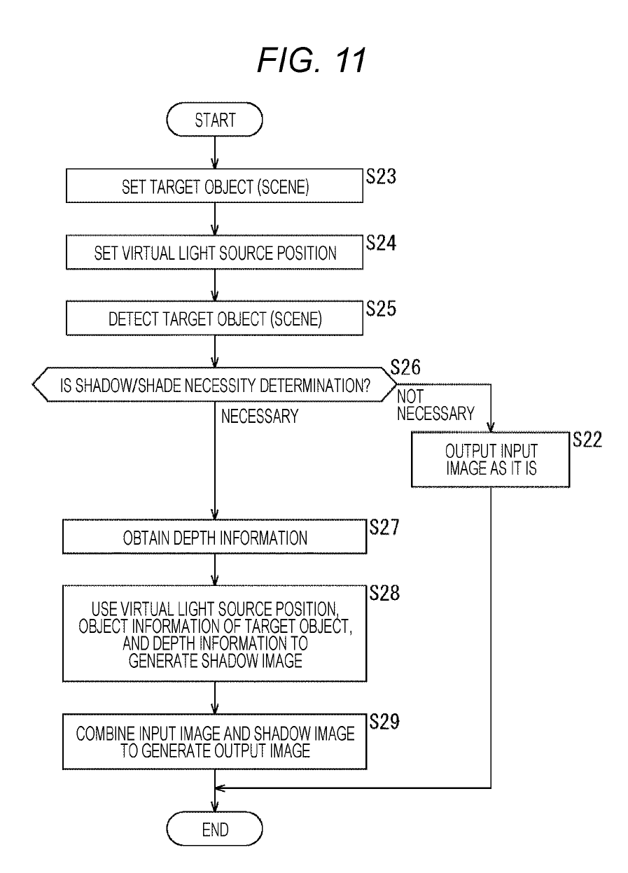

FIG. 11 is a flowchart illustrating exemplary processing of the image processing apparatus 12 in FIG. 10.

Here, note that for simplicity of explanation, it is assumed that the shadow/shade necessity determination unit 41 performs the shadow/shade necessity determination in accordance with the detection information from the object detection unit 62, instead of what is described in FIG. 5.

In step S23, the object setting unit 61 sets the target object and supplies it to the object detection unit 62, and the processing proceeds to step S24.

In step S24, similarly to step S13 of FIG. 9, the virtual light source position setting unit 51 sets the virtual light source position and supplies it to the shadow image generation unit 53, and the processing proceeds to step S25.

In step S25, the object detection unit 62 detects the target object from the input image, then supplies the detection information indicating the detection result to the shadow/shade necessity determination unit 41, and thereafter, the processing proceeds to step S26.

In step S26, the shadow/shade necessity determination unit 41 performs shadow/shade necessity determination of determining whether shadow/shade processing is necessary for the input image from the camera 22 on the basis of the detection information from the object detection unit 62.

In a case where it is determined in the shadow/shade necessity determination in step S26 that the shadow/shade processing is not necessary for the input image from the camera 22, that is, in a case where the target object has not been detected from the input image, the processing proceeds to step S22. In step S22, similarly to step S12 of FIG. 9, the shadow/shade combining processing unit 42 outputs the input image from the camera as it is to the display apparatus 13 as an output image, and the processing is finished.

In addition, in a case where it is determined in the shadow/shade necessity determination in step S26 that the shadow/shade processing is necessary for the input image from the camera 22, that is, the target object appears in the input image and therefore the target object has been detected from the input image, the object detection unit 62 generates object information of the target object detected from the input image, and supplies the information to the shadow image generation unit 53. Then, the processing proceeds from step S26 to step S27, and the shadow/shade combining processing is performed below.

That is, in step S27, similarly to step S14 in FIG. 9, the depth estimation unit 52 obtains the depth information from the 3D image as the input image from the camera 22, supplies the information to the shadow image generation unit 53, and the processing proceeds to step S28.

In step S28, the shadow image generation unit 53 sets solely the target object specified by the object information from the object detection unit 62 among the subjects appearing in the input image as a target for generation, and generates a shadow image, that is, an image of a shadow of the target object, generated by the virtual light source on the basis of the virtual light source position and the depth information, and supplies the generated image to the combining unit 54. The processing proceeds to step S29.

In step S29, similarly to step S16 in FIG. 9, the combining unit 54 combines the input image from the camera 22 and the shadow image from the shadow image generation unit 53 with each other to generate an output image in which a shadow is added to the medical image, and outputs the image to the display apparatus 13, and then, the processing is finished.

Note that while FIGS. 10 and 11 illustrate a case where the object detection unit 62 detects the target object set by the object setting unit 61, the object detection unit 62 can detect a specific scene (for example, scene of suturing, and the like) that are specific to the scene, and can detect an object specific to the scene (for example, a thread or the like used for suturing in a scene of suturing) as a target object. Then, the shadow/shade combining processing unit 41 can, for example, add a shadow to the target object detected from the specific scene.

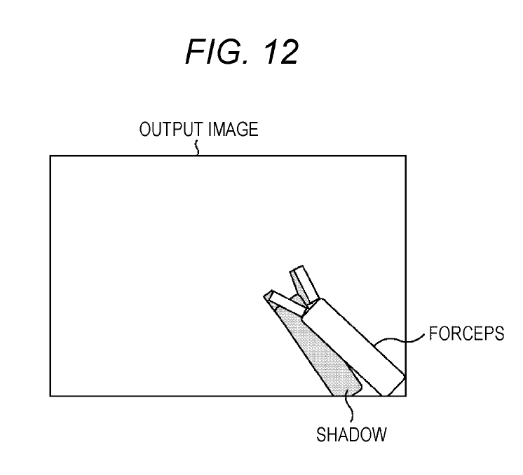

FIG. 12 is a diagram illustrating an exemplary output image obtained by the image processing apparatus 12 of FIG. 10.

That is, FIG. 12 illustrates an exemplary output image in the case where the forceps are set as the target object.

In a case where the forceps are set as the target object, an output image in which a shadow generated by the virtual light source is added to the forceps alone is generated as illustrated in FIG. 12.

According to the output image of FIG. 12, the surgeon naturally (instinctively) can grasp a positional relationship between the forceps and the abdominal wall or the like where the shadow appears using the difference in distance between the forceps appearing on the output image and the shadow of the forceps, for example. Furthermore, the surgeon can naturally grasp the speed of movement of the forceps in the depth direction, for example, by how the shadow of the forceps appearing in the output image changes.

Note that in case of adding the shadow/shade to the forceps or the like, it is allowable to add shadow/shade only to a predetermined range from the distal end portion, rather than the entire forceps or the like. In this case, it is possible to reduce the processing of adding shadow/shade.

As described above, the image processing apparatus 12 of FIG. 10 detects a target object from an input image, and generates an output image obtained by adding a shadow to the target object.

For a target object, it is possible to assume a predetermined thickness typically held by the target object, that is, to estimate the thickness.

Accordingly, instead of obtaining the shadow region sh2 (FIG. 8) with assumption that the target subject is solid (present) toward the back side (the deeper side) of the target subject as viewed from the camera 22, as described with reference to FIGS. 7 and 8, the shadow image generation unit 53 of FIG. 10 can obtain the shadow region sh1 (FIG. 7) similar to the shadow that is supposed to be generated to the target object with assumption that there is a target object having merely an estimated value on the basis of the estimated value of the thickness of the target object.

With this configuration, it is possible to prevent the occurrence of the shadow region sh2 that is not supposed to appear, in the output image as an artifact.

Note that the image processing apparatus 12 illustrated in FIG. 10 can also perform addition and suppression of the shadows as shadow/shade processing similarly to the case of FIG. 5.

<Third Exemplary Configuration of Image Processing Apparatus 12>

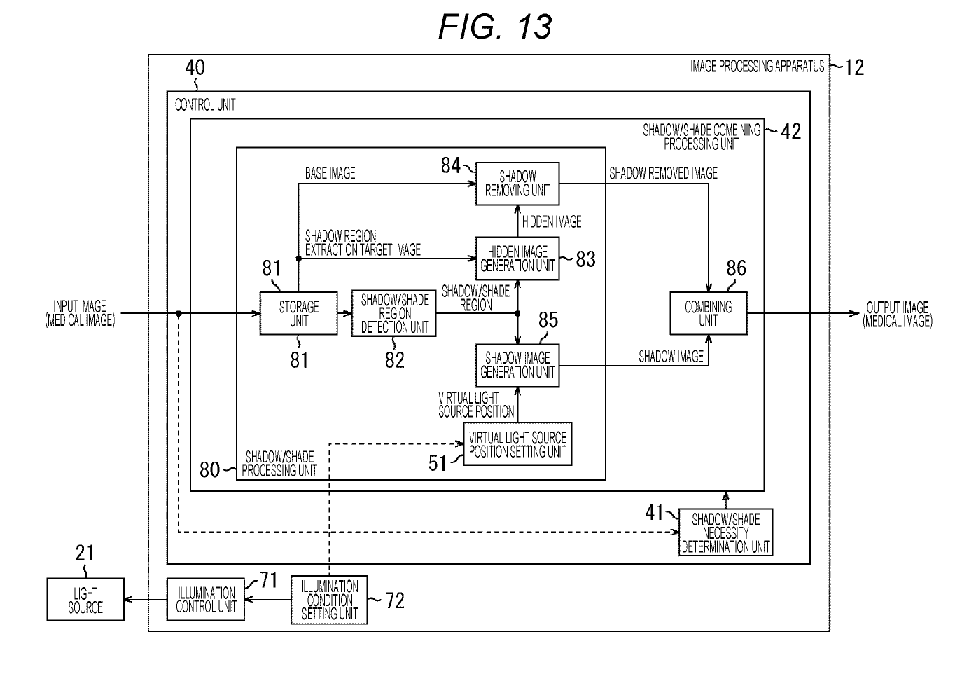

FIG. 13 is a block diagram illustrating a third exemplary configuration of the image processing apparatus 12 in FIG. 2.

Note that in the figure, portions corresponding to the case of FIG. 5 are denoted by the same reference numerals, and the description thereof will be omitted as appropriate below.

In FIG. 13, the image processing apparatus 12 includes the control unit 40, an illumination control unit 71, and an illumination condition setting unit 72.

The control unit 40 includes the shadow/shade necessity determination unit 41 and the shadow/shade combining processing unit 42.

The shadow/shade combining processing unit 42 includes a shadow/shade processing unit 80 and a combining unit 86.

The shadow/shade processing unit 80 includes the virtual light source position setting unit 51, a storage unit 81, a shadow/shade region detection unit 82, a hidden image generation unit 83, a shadow removing unit 84, and a shadow image generation unit 85.

This allows the image processing apparatus 12 of FIG. 13 to have a similar configuration of FIG. 5 in that it includes the control unit 40, and that the control unit 40 includes the shadow/shade necessity determination unit 41 and the shadow/shade combining processing unit 42.

The image processing apparatus 12 of FIG. 13, however, is different from the case of FIG. 5 in that the illumination control unit 71, and the illumination condition setting unit 72 are newly provided.

Moreover, the image processing apparatus 12 of FIG. 13 has a configuration in which the shadow/shade combining processing unit 42 includes the shadow/shade processing unit 80 and the combining unit 86, and in this respect, is different from the case of FIG. 5 in which the shadow/shade combining processing unit 42 includes the shadow/shade processing unit 50 and the combining unit 54.

The image processing apparatus 12 illustrated in FIG. 13 detects a shadow/shade region having a shadow/shade in an input image using a plurality of frames of an input image photographed under a plurality of different (setting) illumination conditions described below, and performs shadow/shade processing on the shadow/shade region in accordance with the virtual light source position.

The illumination control unit 71 controls the light source 21 so as to change the illumination conditions of the illumination by the light source 21, that is, the illumination conditions in illuminating the subject such as the surgical site in accordance with the illumination conditions supplied from the illumination condition setting unit 72.

Here, examples of the illumination conditions include the position of the light source 21, the intensity and direction of the illumination light emitted from the light source 21, and the like.

The illumination condition setting unit 72 sets a plurality of predetermined different illumination conditions in accordance with user's operation or the like and supplies the set illumination conditions to the illumination control unit 71.

Note that the illumination conditions set by the illumination condition setting unit 72 will be also referred to as set illumination conditions.

The illumination control unit 71 periodically selects each of the plurality of setting illumination conditions from the illumination condition setting unit 72 as an illumination condition of interest, and controls the light source 21 such that the illumination condition for illuminating the subject becomes the illumination condition of interest.

An input image (medical image) from the camera 22 is supplied to the storage unit 81.

Here, in FIG. 13, a 2D image is photographed by the camera 22, and the 2D image is supplied as an input image from the camera 22 to the storage unit 81. Alternatively, the input image photographed by the camera 22 may be a 3D image similarly to the case of the image processing apparatus 12 in FIGS. 5 and 10.

The storage unit 81 sequentially stores frames of the input image supplied from the camera 22.

Here, in FIG. 13 as described above, the illumination control unit 71 periodically selects each of the plurality of setting illumination conditions as an illumination condition of interest, and controls the light source 21 such that the illumination condition for illuminating the subject becomes the illumination condition of interest.

Therefore, the camera 22 repeats continuous photographing an input image (frames of the image) under each of a plurality of setting illumination conditions.

For example, in a case where the plurality of setting illumination conditions corresponds to two different setting illumination conditions, successive photographing of the frames of the input image are repeated under the two different setting illumination conditions. Moreover, for example, in a case where the plurality of setting illumination conditions corresponds to three different setting illumination conditions, successive photographing of the frames of the input image are repeated under the three different setting illumination conditions.

Now, a plurality of frames of an input image successively photographed under each of a plurality of (different) setting illumination conditions set by the illumination condition setting unit 72 will be referred to as a frame set.

The storage unit 81 has at least a storage capacity for storing input images corresponding to the number of frames constituting the frame set.

The storage unit 81 supplies the frame set stored in the storage unit 81 to the shadow/shade region detection unit 82.

In addition, the storage unit 81 selects a base image and a shadow region extraction target image to be described below from the frame set. Then, the storage unit 81 supplies the base image to the shadow removing unit 84 and together with this, supplies the shadow region extraction target image to the hidden image generation unit 83.

The shadow/shade region detection unit 82 uses the frame set from the storage unit 81, that is, a plurality of frames of the input image successively photographed under each of the plurality of different setting illumination conditions to detect a shadow/shade region having shadow/shade in the input image, and then, supplies the input image in which the shadow/shade region is specified to the hidden image generation unit 83 and the shadow image generation unit 85.

The hidden image generation unit 83 uses the shadow region extraction target image from the storage unit 81 and the shadow/shade region (input image in which the shadow shade region is specified) from the shadow/shade region detection unit 82 so as to generate an image in which a portion that is a shadow region that is invisible hidden by the shadow in the base image but appears in the shadow region extraction target image is specified, as a hidden image, and then, supplies the generated hidden image to the shadow removing unit 84.

The shadow removing unit 84 combines the hidden image from the hidden image generation unit 83 to the base image from the storage unit 81 so as to generate an image in which a portion that used to be invisible as a shadow region in the base image is now visible as a shadow removed image obtained by removing the shadow region from the base image, and supplies the generated image to the combining unit 86.

The shadow image generation unit 85 receives the input image in which the shadow/shade region is specified supplied from the shadow/shade region detection unit 82 and receives the virtual light source position supplied from the virtual light source position setting unit 51.

The shadow image generation unit 85 uses the input image in which the shadow/shade region supplied from the shadow/shade region detection unit 82 to obtain a shadow image in which the shadow region to be added to the base image is specified.

Furthermore, the shadow image generation unit 85 generates a new shadow image by adding a new shadow (region) to the shadow region of the shadow image in accordance with the virtual light source position from the virtual light source position setting unit 51, and supplies the generated image to the combining unit 86.

The combining unit 86 combines the shadow removed image from the shadow removing unit 84 and the (new) shadow image (shadow region) from the shadow image generation unit 85 with each other similarly to the case of the combining unit 54 in FIG. 5, for example, so as to generate an output image to which a new shadow has been added, and outputs the generated image to the display apparatus 13.

Here, the shadow removed image or the combined image obtained by combining the shadow removed image and the shadow image with each other can be considered as the shadow/shade corrected image (image obtained by performing shadow/shade related correction to the input image) described in FIG. 5.

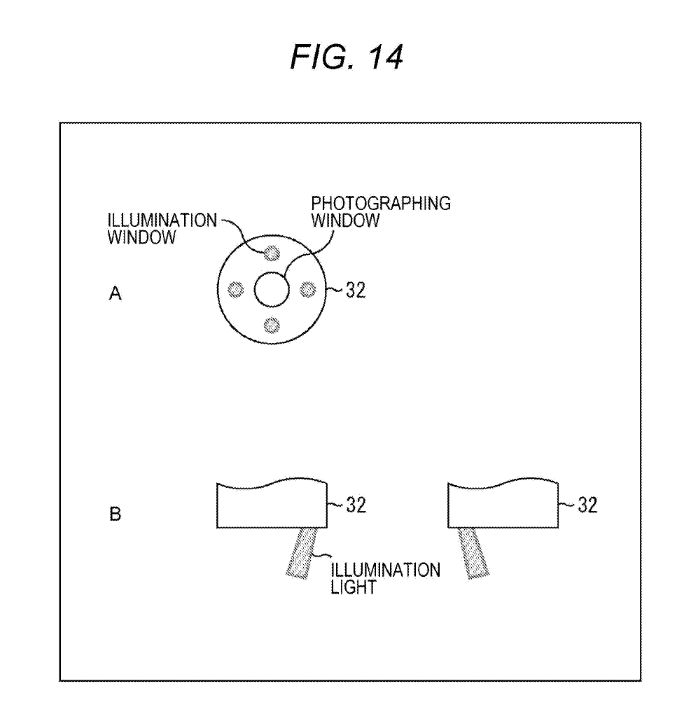

FIG. 14 is a diagram illustrating an example of control of the light source 21 in the illumination control unit 71.

A of FIG. 14 is a front view illustrating an exemplary configuration of a distal end of the endoscope scope 32 constituting the endoscope 11 in a case where the distal end is defined as the front.

B of FIG. 14 is a side view illustrating an exemplary configuration of the distal end of the endoscope scope 32.

In A of FIG. 14, a photographing window and an illumination window are provided at the distal end of the endoscope scope 32.

Reflected light from the subject is incident from the photographing window to be led to the camera 22.

Note that in FIG. 14, the front surface of the distal end of the endoscope scope 32 is formed in a (substantially) circular shape, and the photographing window is provided in a central portion of the circle.

The illumination window is a portion of the light source 21, and illumination light is applied (emitted) from the illumination window.

Note that FIG. 14 is an example in which four illumination windows are provided around the photographing window. The number of illumination windows, however, is not limited to four. That is, the endoscope scope 32 can include a plurality of illumination windows other than four.

Illumination light is applied from the illumination window under the control of the illumination control unit 71. For example, the illumination control unit 71 can control (select) the illumination window used to apply the illumination light from among the four illumination windows.

For example, the illumination condition setting unit 72 can set the two setting illumination conditions such that illumination light is to be applied from the right illumination window among the four illumination windows at the time of photographing an input image of one of the odd frame and the even frame, and that illumination light is to be applied from the left illumination window among the four illumination windows at the time of photographing an input image of the other of the odd frame and the even frame.

A shadow is generated on the left side of the subject by illumination light applied from the right illumination window in one of the odd frame and the even frame of the input image, and a shadow is generated on the right side of the subject by illumination Light applied from the left illumination window in the other frame.

Note that in addition to the position of the illumination light (illumination window through which the illumination light is applied), the illumination direction of the illumination light, the intensity of the illumination light, or the like can be set as the setting illumination conditions.

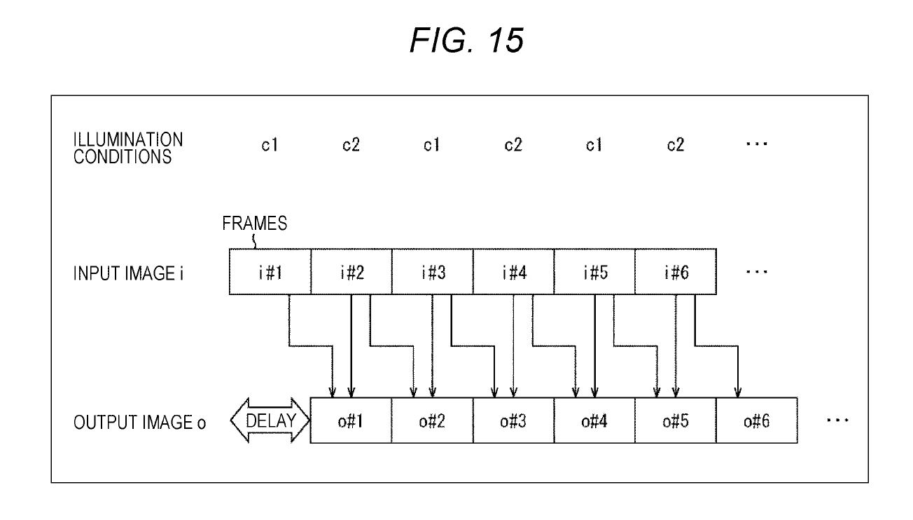

FIG. 15 is a diagram illustrating a first example of generation of an output image from a frame of an input image photographed under each of a plurality of (setting) illumination conditions.

Note that for simplicity of explanation, the following description assumes that the illumination control unit 71 sequentially selects each of the plurality of setting illumination conditions from the illumination condition setting unit 72 as an illumination condition of interest, frame by frame, for example. That is, the illumination conditions for illuminating the subject is assumed to be switched frame by frame.

In FIG. 15, the illumination condition for illuminating the subject is periodically switched to each of two setting illumination conditions c1 and c2 on a frame basis in photographing the input image i.

That is, odd-numbered frames i#2n−1 (n=1, 2, . . . ) of an input image i are photographed under the setting illumination condition c1 while even-numbered frames i#2n are photographed under the setting illumination condition c2.

In this case, the two successively photographed frames i#k and i#k+1 (k=1, 2, . . . ) of the input image i are defined as the frame set described with reference to FIG. 13 to be generated as a frame o#k of an output image o.

That is, when it is assumed that the latest frame of the input image i is the frame i#k+1, two consecutive frames of the input image, that is, the frame i#k+1 and the frame i#k one frame before the frame i#k+1 are used to generate the latest frame o#k of the output image o.

As described above, in a case where the illumination condition for illuminating the subject is switched to each of the two setting illumination conditions c1 and c2 frame by frame, a frame set of the latest frame i#k+1 of the input image i and the frame i#k one frame before that frame set is needed in order to generate the frame o#k of the output image o.

For this reason, there is a delay of one frame before the start of the output of the output image o after the start of the photographing of the input image i.

Since endoscopic surgery needs viewing of real-time images, it is important to minimize the occurrence of delay during the time after the start of the photographing of the input image i before the start of the output of the output image o.

Accordingly, in FIG. 15, with respect to the frame i#1 which is first obtained after the start of the photographing of the input image i, the frame i#1 can be output as it is as the output image o (a frame of the output image o). In this case, the output image o is not an image that has undergone the shadow/shade (combining) processing, while it is possible to prevent an occurrence of delay during the time after the start of the photographing of the input image i before the start of the output of the output image o.

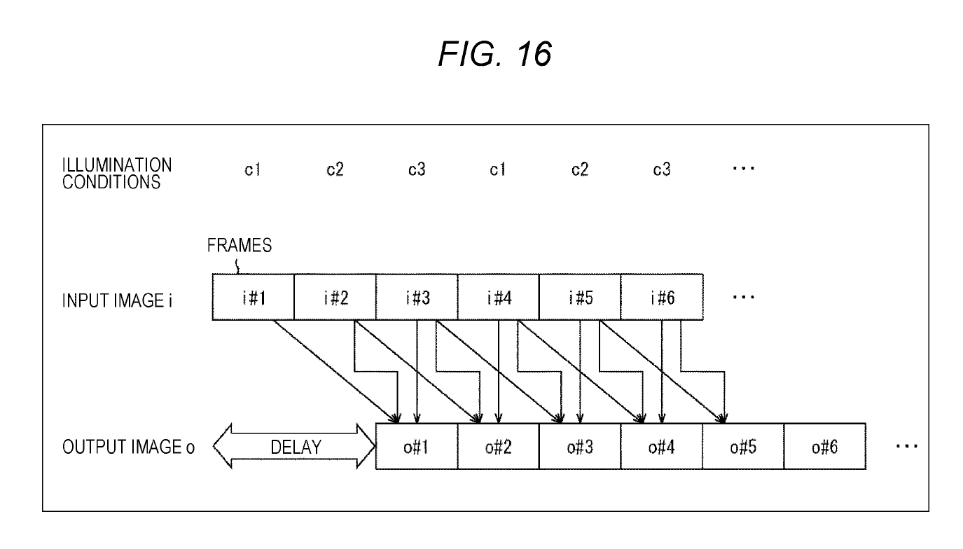

FIG. 16 is a diagram illustrating a second example of generation of an output image from a frame of an input image photographed under each of a plurality of (setting) illumination conditions.

In FIG. 16, the illumination condition for illuminating the subject is periodically switched to each of three setting illumination conditions c1, c2, and c3 on a frame basis in photographing the input image i.

That is, the 3n−2 th frame of the input image i, namely a frame i#3n−2 is photographed under the setting illumination condition c1, the 3n−1 th frame of the input image i, namely a frame i#3n−1 is photographed under the setting illumination condition c2, and the 3n th frame of the input image i, namely a frame i#3n is photographed under the setting illumination condition c3.

In this case, the three successively photographed frames i#k to i#k+2 of the input image i are defined as the frame set described with reference to FIG. 13 to be generated as a frame o#k of the output image o.

That is, when it is assumed that the latest frame of the input image i is the frame i#k+2, three latest frames i#k to i#k+2 of the input image i including the frame i#k+2 are used to generate the latest frame o#k of the output image o.

As described above, in a case where the illumination condition for illuminating the subject is switched to each of the three setting illumination conditions c1 to c3 frame by frame, a frame set of the three frames i#k to i#k+2 of the input image i is needed in order to generate the frame o#k of the output image o.

For this reason, there is a delay of two frames before the start of the output of the output image o after the start of the photographing of the input image

As described with reference to FIG. 15, it is important to achieve endoscopic surgery that minimizes the occurrence of delay during the time after the start of the photographing of the input image i before the start of the output of the output image o.

Therefore, in a case where the illumination condition for illuminating the subject is switched to each of the three setting illumination conditions c1 to c3 to photograph the input image i, the output image can be generated as follows.

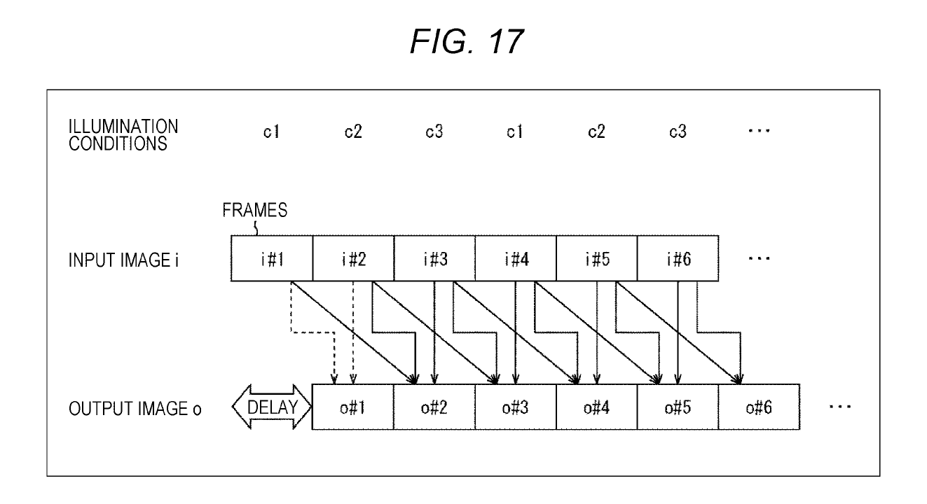

That is, FIG. 17 is a diagram illustrating a third example of generation of an output image from a frame of an input image photographed under each of a plurality of (setting) illumination conditions.

Similarly to FIG. 16, FIG. 17 is a case where the illumination condition for illuminating the subject is periodically switched to each of three setting illumination conditions c1, c2, and c3 on a frame basis in photographing the input image i.

Then, as in the case of FIG. 16, as for the second and subsequent frames o#k of the output image o, three successively photographed frames i#k to i#k+2 of the input image i are defined as a frame set so as to generate the frame o#k of the output image o.

However, as in the case of FIG. 15, as for the first frame o#1 of the output image o, two successively photographed frames i#k and i#k+1 of the input image i are defined as a frame set so as to generate the frame o#k of the output image o.

In this case, it is possible to suppress the delay from the start of the photographing of the input image i before the start of output of the output image o to a delay of one frame.

Note that, in FIG. 17, with respect to the frame i#1 which is first obtained after the start of the photographing of the input image i, the frame i#1 can be output as it is as the output image o (a frame of the output image o). In this case, the output image o is not an image that has undergone the shadow/shade processing, while it is possible to prevent an occurrence of delay during the time after the start of the photographing of the input image i before the start of the output of the output image o.

In addition, the number of illumination conditions for illuminating the subject is not limited to the above two or three, and may be four or more.

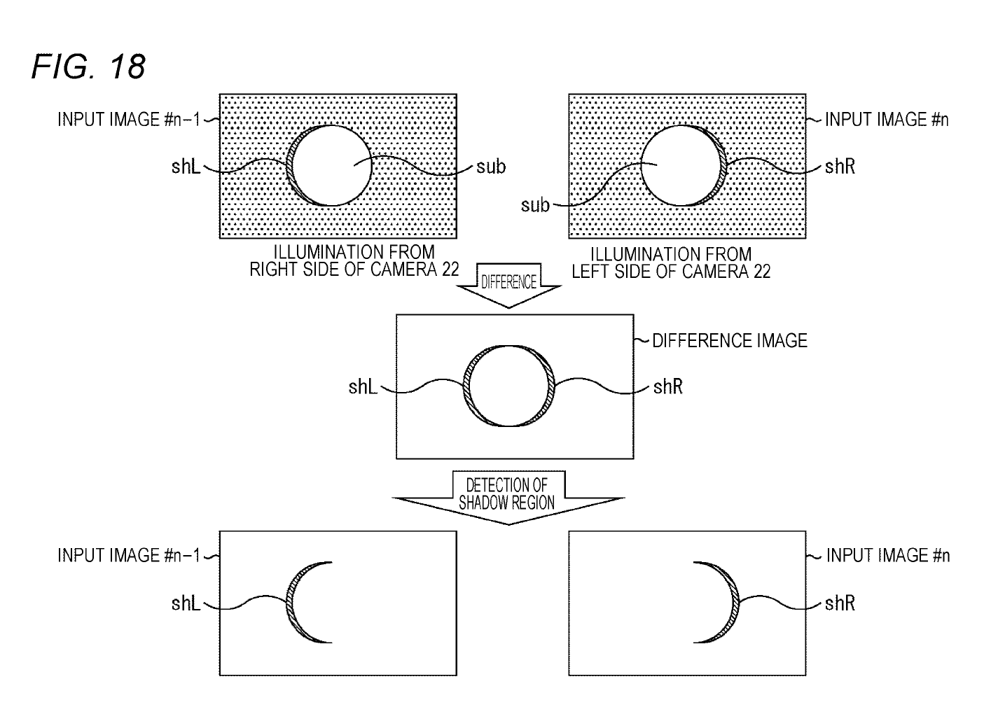

FIG. 18 is a diagram illustrating exemplary processing of the shadow/shade region detection unit 82 in FIG. 13.

Note that for simplicity of explanation, the following description assumes that the illumination condition for illuminating the subject is periodically switched alternately to each of the two setting illumination conditions on a frame basis so as to photograph the input image i.

Furthermore, illumination of the subject from the position on the right side of the camera 22 (position on the right side of the camera 22 the subject is viewed from the camera 22) is adopted as one set illumination condition among the two set illumination conditions, while illumination of the subject from the position on the left side of the camera 22 is adopted as the other illumination condition.

For example, when it is assumed that the latest frame #n (hereinafter also referred to as an input image #n) of the input image is photographed by illuminating a subject sub from the position on the left side of the camera 22, the latest input image #n and the input image (frame of the input image) #n−1 photographed immediately before the input image #n are supplied from the storage unit 81 to the shadow/shade region detection unit 82 as a frame set.

Note that, the subject sub is illuminated from the position on the left side of the camera 22 in the latest input image #n, while the subject sub is illuminated from the position on the right side of the camera 22 in the input image #n−1 photographed immediately before the input image #n.

Therefore, in the input image #n in which the subject sub is illuminated from the position on the left side of the camera 22, there is a shadow region shR of the shadow generated by the subject sub on the right side of the subject sub. Moreover, in the input image #n−1 in which the subject sub is illuminated from the position on the right side of the camera 22, there is a shadow region shL of the shadow generated by the subject sub on the left side of the subject sub.

The shadow/shade region detection unit 82 obtains information related to the difference such as the difference absolute value units of pixels between the input images #n and #n−1, and generates a difference image having the difference absolute value as the pixel value.

Furthermore, the shadow/shade region detection unit 82 detects all the regions formed by collection of pixels having large pixel values in the difference image, and detects a region having a predetermined area or more from the regions as a candidate of a shadow/shade region (region in which shadow/shade appears). Note that the shadow/shade region detection unit 82 can detect all the regions formed by collection of pixels having large pixel values in the difference image as the candidate of the shadow/shade region.

In the input image #n, there is no shadow in the region corresponding to the shadow region shL of the input image #n−1. In the input image #n−1, there is no shadow in the region corresponding to the shadow region shR of the input image #n.

Therefore, the pixel value (absolute value of difference) of the pixels of the shadow regions shL and shR becomes large in the difference image between the input images #n and #n−1, and thus, the shadow regions shL and shR are detected as candidates for the shadow/shade region.

After detecting the candidate of the shadow/shade region, the shadow/shade region detection unit 82 obtains average luminance, that is, an average value of the luminance of the pixels within the candidates of the shadow region for each of the input images #n and #n−1.

Then, the shadow/shade region detection unit 82 detects candidates of the shadow/shade region having average luminance that is luminance threshold or below as a shadow region (region of shadow) as a type of the shadow/shade region for each of the input images #n and #n−1, and then, supplies the input images #n and #n−1 in each of which a shadow region has been specified to the hidden image generation unit 83 and the shadow image generation unit 85.

In FIG. 18, the shadow region shR of the input image #n and the shadow region shL of the input image #n−1 are detected, and then, the input image #n in which the shadow image shR has been specified and the input image #n−1 in which the shadow image shL has been specified are supplied from the shadow region detection unit 82 to the hidden image generation unit 83 and the shadow image generation unit 85.

Note that a predetermined fixed value can be adopted as the luminance as the threshold used in the shadow/shade region detection unit 82, for example. Furthermore, the luminance as the threshold can be determined in accordance with the histogram of the entire input images #n and #n−1 or of the luminance of the candidate of the shadow/shade region, for example.

In addition, while FIG. 18 is a case where the candidates of the shadow/shade region having average luminance that is the luminance as the threshold are detected as the shadow region as a type of the shadow/shade region for each of the input images #n and #n−1, it is also possible to detect, for example, a candidate for a shadow/shade region having average luminance that is not the luminance as the threshold or below as a shade region (region of shade) that is another type of the shadow/shade region.

In addition, while the shade region can be processed similarly to the shadow region, the explanation of the processing for the shade region will be omitted here for simplicity of explanation.

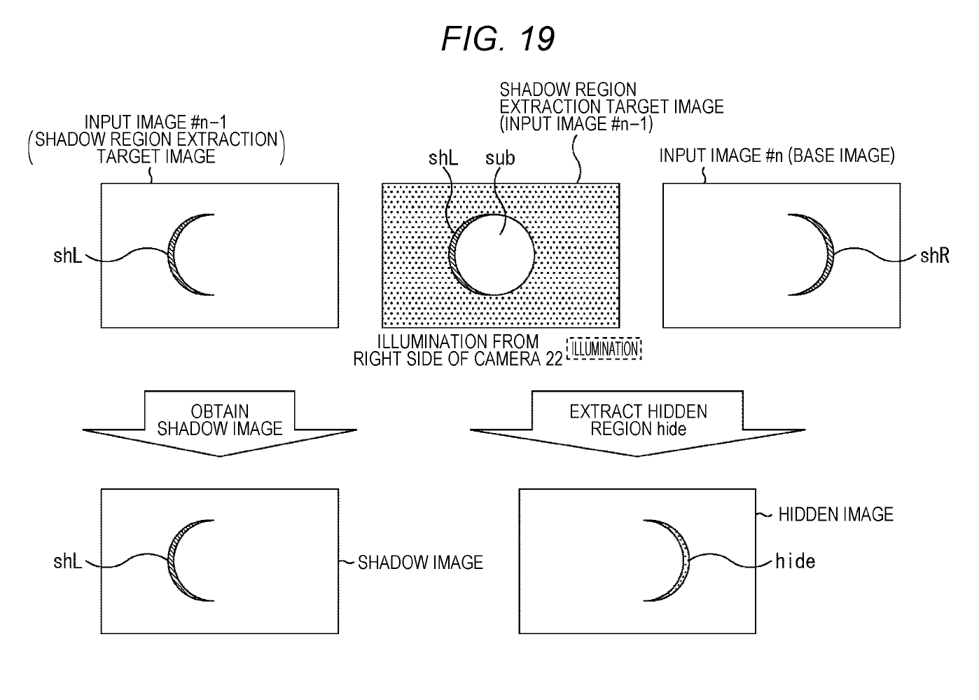

FIG. 19 is a diagram illustrating exemplary processing of the hidden image generation unit 83 and the shadow image generation unit 85 illustrated in FIG. 13.

As described with reference to FIG. 13, the storage unit 81 selects a base image and a shadow region extraction target image from a frame set, that is, from the input images #n and #n−1 in the target case.

The base image is an image as a base of an output image, and the latest input image among the input images of the frame set is selected as the base image. Accordingly, for the frame set of the input images #n and #n−1, the latest input image #n is selected as the base image.

The shadow region extraction target image is an image from which a shadow region to be a source of a shadow (region) to be added to an output image is extracted (detected). An input image having the illumination (light source) position closest to the virtual light source position among the input images of the frame set is selected as the shadow region extraction target image.

For example, it is assumed that the virtual light source position is set at a position at a position in a direction opposite to the left side position of the camera 22 from the right side position of the camera 22 (that is the position on more rightward than the right side of the camera 22) on a line connecting a position on the left side of the camera 22 as the position of the illumination (the position of the light source (illumination window) emitting the illumination light for illuminating the subject) when the input image #n is currently photographed with the position on the right side of the camera 22 as the position of the illumination when the input image #n−1 is photographed.

In this case, the position on the right side of the camera 22 is closer to the virtual light source position among the position on right side of the camera 22 among the position on the left side of the camera 22 as the position of the illumination when the input image #n is photographed and the position on the right side of the camera 22 as the position of the illumination when the input image #n−1 is photographed.

Therefore, the input image #n−1 having the illumination position at the time of photographing on the right side of the camera 22 is selected as the shadow region extraction target image.

The shadow image generation unit 85 obtains the input image #n−1 as the shadow region extraction target image among the input images #n−1 and #n in which the shadow regions shL and shR as the shadow/shade regions supplied from the shadow/shade region detection unit 82 are respectively specified, as a shadow image in which the shadow region to be added to the base image specified.

Meanwhile, the hidden image generation unit 83 extracts a region corresponding to the shadow region shR of the input image #n he that is a base image among the input images #n and 4n−1 supplied from the shadow/shade region detection unit 82 from the input image 4n−1 that is the shadow region extraction target image supplied from the storage unit 81.

That is, the hidden image generation unit 83 extracts the region corresponding to the shadow region shR of the input image #n as the base image from the input image #n−1 which is the shadow region extraction target image, as a hidden region hide that is the shadow region shR invisible behind a shadow in the base image but appears in the shadow region extraction target image.

Then, the hidden image generation unit 83 supplies the input image #n−1 that is the shadow region extraction target image in which the hidden region hide is specified, to the shadow removing unit 84 as a hidden image.

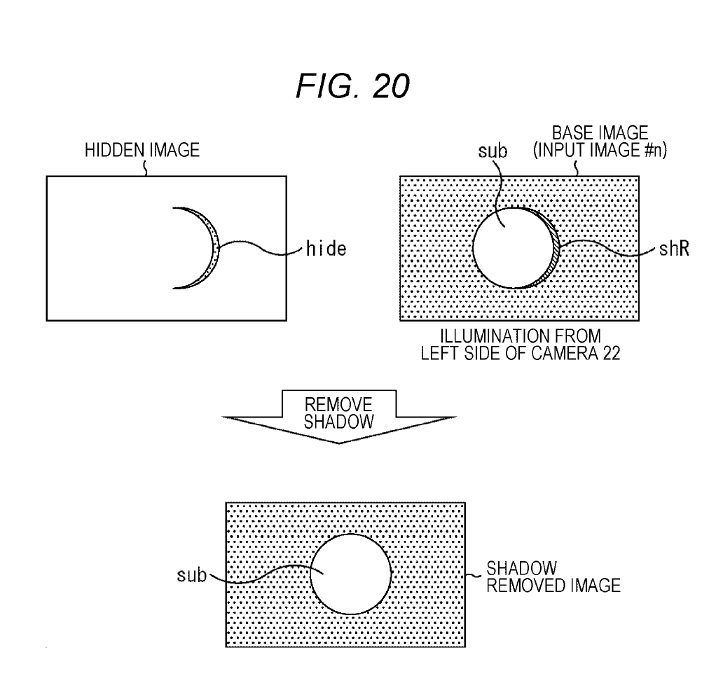

FIG. 20 is a diagram illustrating exemplary processing of the shadow removing unit 84 in FIG. 13.

The shadow removing unit 84 combines the hidden region hide of the hidden image from the hidden image generation unit 83 to input image #n as the base image from the storage unit 81 so as to generate an image in which a portion that used to be invisible as the shadow region shR in the base image is now visible as a shadow removed image obtained by removing the shadow region from the base image, and supplies the generated image to the combining unit 86.

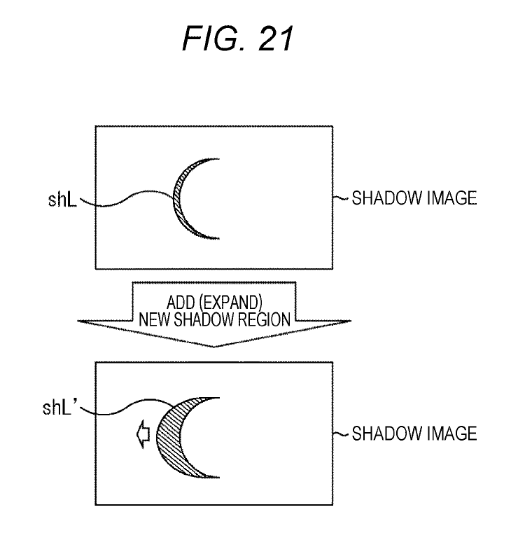

FIG. 21 is a diagram illustrating exemplary processing of the shadow image generation unit 85 in FIG. 13.

As described with reference to FIG. 19, the shadow image generation unit 85 obtains the input image #n−1 which is the shadow region extraction target image in which the shadow region shL is specified, as a shadow image.

While the shadow region shL of this shadow image is the shadow region shL of the input image #n−1 being the shadow region extraction target image, the shadow image generation unit 85 generates new shadow image obtained by adding a new shadow to the shadow region shL of the shadow image in accordance with the virtual light source position from the virtual light source position setting unit 51

That is, the shadow image generation unit 85 expands the contour of the predetermined direction of the shadow region shL of the shadow image by a predetermined size (the number of pixels) in a predetermined direction while maintaining the shape of the contour, thereby expanding the shadow region shL to a new shadow region shL′ that appears as a region obtained by adding a new shadow to the shadow region shL.

Here, the predetermined direction and size in expanding the contour of the shadow region shL are determined in accordance with the positional relationship between the position of the illumination (light source) at the time of photographing the input image #n−1 being the shadow region extraction target image and virtual light source position.

That is, the predetermined direction of expanding the contour of the shadow region shL is determined to be the direction from the virtual light source position toward the position of the illumination at the time of photographing of the input image #n−1 being the shadow region extraction target image.

Here, as described in FIG. 18, the position of the illumination at the time of photographing the input image #n−1 being the shadow region extraction target image is the position on the right side of the camera 22, while the virtual light source position is set to a position further rightward than the right side position of the camera 22.

Therefore, the predetermined direction of expanding the contour of the shadow region shL is determined to be the left direction (the left direction when the subject is viewed from the camera 22).

In addition, the predetermined size/direction of expanding the contour of the shadow region shL is determined to be a value corresponding to a distance between the virtual light source position and the position of the illumination at the time of photographing the input image #n−1 being the shadow region extraction target image that is, a value proportional to the distance, for example.

Therefore, the more distant (rightward) the virtual light source position is located from the position of the illumination at the time of photographing the input image #n−1 being the shadow region extraction target image, the more expanded the contour of the shadow region shL is.

Note that the extension of the shadow region shL of the shadow image is performed by changing the pixel value of the pixel of the shadow image to a pixel value representing the shadow.

Examples of the pixel value representing the shadow that can be adopted include black, a dark color, a color selected (set) by for easy identification by the user, and the like.

Moreover, it is also possible to adopt as the pixel value representing the shadow, a pixel value obtained by alpha blending (alpha blending using a number a larger than 0.0 and smaller than 1.0) using, for example an original pixel value of the pixel and the black color, dark color, color selected for easy identification by the user, and the like.

Even in a case where a shadow appears in an image, the subject in that shadow can be visually recognized with clarity according to the density (thinness) of the shadow. In this sense, the image in which the subject in the shadow is completely invisible (not appearing) might be an unnatural or artificial image.

As described above, with the adoption of the pixel value obtained by alpha blending the original pixel value of the pixel and black or the like as the pixel value representing a shadow, it is possible to suppress generation of the output image that is an unnatural image as described above.

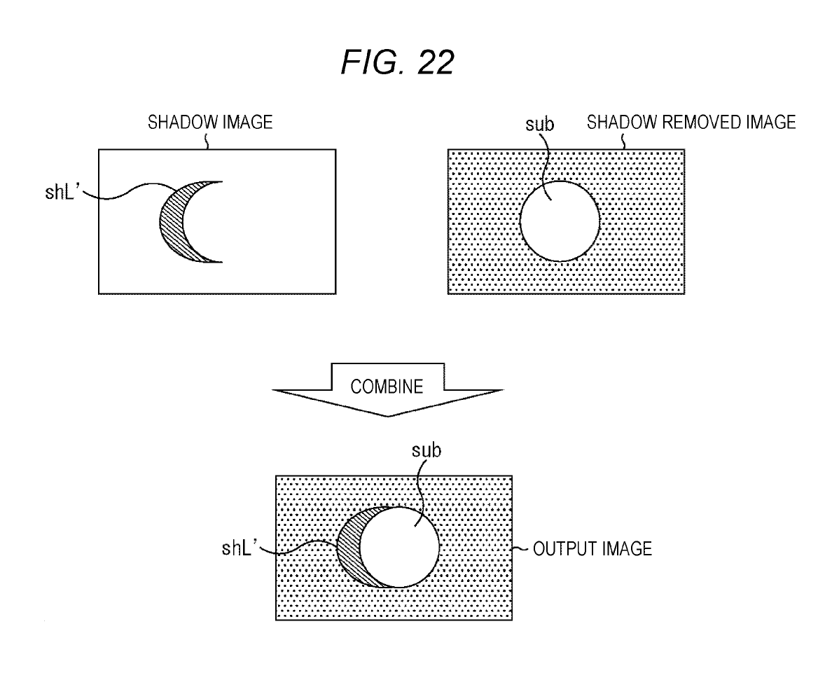

FIG. 22 is a diagram illustrating exemplary processing of the combining unit 86 in FIG. 13.

The combining unit 86 combines the shadow removed image from the shadow removing unit 84 and the (new) shadow image (shadow region shL′ in the shadow image) from the shadow image generation unit 85 by alpha blending, for example, to generates an output image to which the new shadow region shL′ of the shadow image has been added as a shadow of the subject sub, and outputs the image to the display apparatus 13.

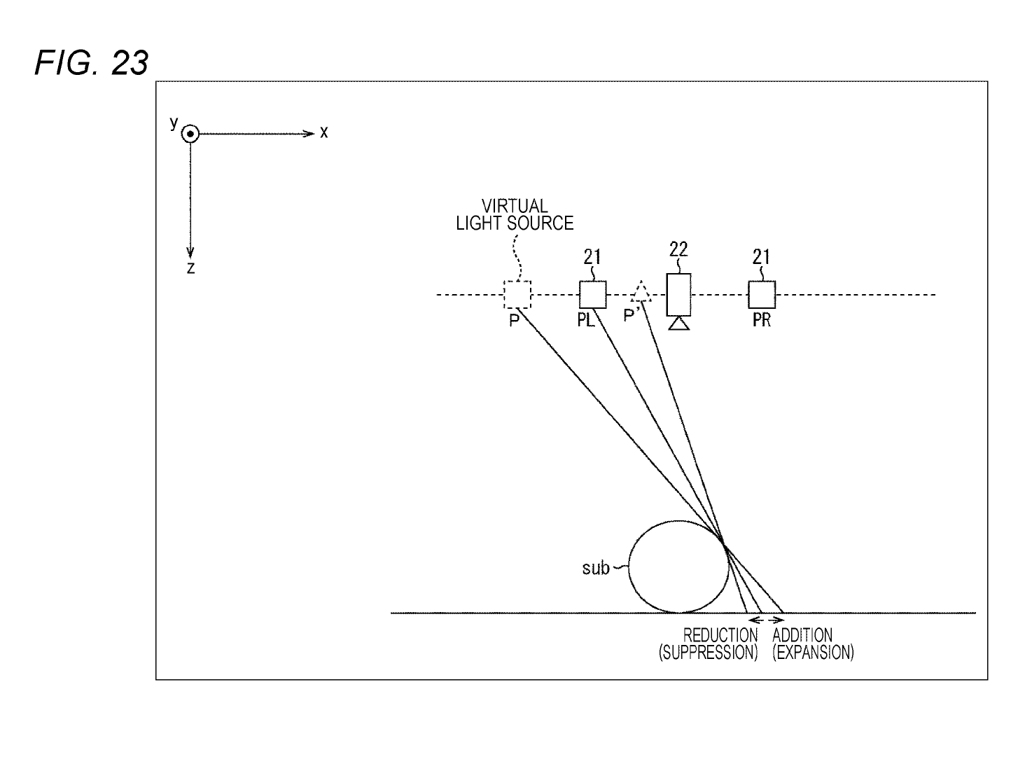

FIG. 23 is a diagram illustrating an example of the virtual light source position set by the virtual light source position setting unit 51 in FIG. 13.

In FIG. 23, the (actual) light source 21 exists at a position PR on the right side of the camera 22 and a position PL on the left side of the camera 22, and the subject sub is illuminated from each of the positions PR and PL alternately on a frame-by-frame basis as described with reference to FIG. 18.

In the case of adding a shadow, a virtual light source position P is set at a position outer than the positions PR and PL when viewed from the subject sub. This is because with the setting the virtual light source position P at a position P′ inner than the positions PR and PL rather than outer than the positions PR and PL, the shadow of the subject sub would be reduced, rather than expanded.

Note that, in the case of suppressing the shadow, the virtual light source position P is set at a position inner than the positions PR and PL when viewed from the subject sub.

Here, in a case where there is no need to specify whether to add a shadow or suppress the shadow as shadow/shade (combining) processing, the virtual light source position setting unit 51 can set a virtual light source position P at an arbitrary position, for example.

In contrast, as described above, the virtual light source position P needs to be set at a position outside the portion between the position PR and PL of the light source 21 in a case of adding a shadow, and the virtual light source position P needs to be set at a position inside the portion between the position PR and PL of the light source 21 in a case of suppressing the shadow. Therefore, in this case, the virtual light source position setting unit 51 needs to recognize the positions PR and PL of the light source 21. The virtual light source position setting unit 51 can recognize the positions PR and PL of the light source 21 from the illumination condition set by the illumination condition setting unit 72 (FIG. 13), for example.

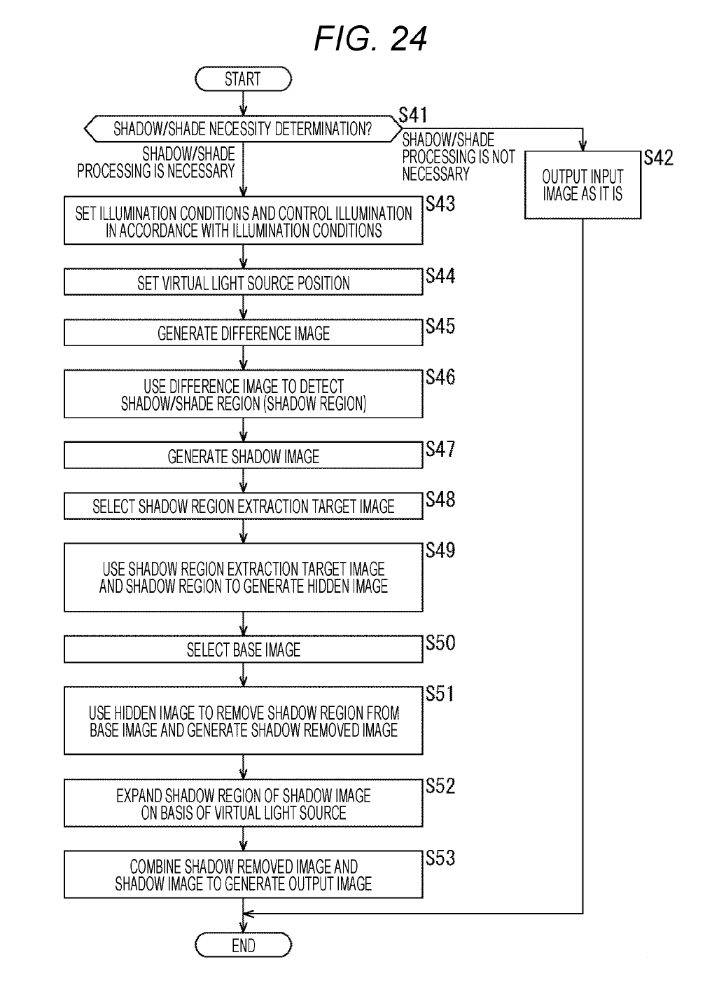

FIG. 24 is a flowchart illustrating exemplary processing of the image processing apparatus 12 in FIG. 13.

In step S41, the shadow/shade necessity determination unit 41 performs shadow/shade necessity determination.

In a case where it is determined in the shadow/shade necessity determination in step S41 that the shadow/shade processing is not necessary for the input image from the camera 22, the processing proceeds to step S42, and then, the shadow/shade combining processing unit 42 outputs the input image from the camera as it is to the display apparatus 13 as an output image, and the processing is finished.

In another case where it is determined in the shadow/shade necessity determination in step S41 that the shadow/shade processing is necessary for the input image from the camera 22, the processing proceeds to step S43, and then, the illumination condition setting unit 72 sets a plurality of setting illumination conditions and supplies the set conditions to the illumination control unit 71.

The illumination control unit 71 periodically selects each of the plurality of setting illumination conditions from the illumination condition setting unit 72 as an illumination condition of interest, and starts processing of controlling the light source 21 (illumination by the light source 21) such that the illumination condition for illuminating the subject becomes the illumination condition of interest.

With this configuration, medical images obtained by photographing under illumination of the subject with a plurality of illumination conditions are sequentially supplied an input image from the camera 22 to the storage unit 81 and stored there.

Here, note that for simplicity of explanation, it is assumed that two setting illumination conditions are alternately switched as the illumination conditions for the subject as described with reference to FIG. 18, for example. In this case, the frame set includes two frames of the latest frame and the immediately preceding frame of the input image.

Thereafter, the processing proceeds from step S43 to step S44. The virtual light source position setting unit 51 sets the virtual light source position and supplies the position to the shadow image generation unit 85, and the processing proceeds to step S45.

In step S45, as described in FIG. 18, the shadow/shade region detection unit 82 generates a difference image of two frames of the input image as the frame set stored in the storage unit 81, and the processing proceeds to step S46.

In step S46, as described with reference to FIG. 18, the shadow/shade region detection unit 82 detects a shadow region as a shadow/shade region having a shadow/shade in the input image as a frame set using the difference image. Then, the shadow/shade region detection unit 82 supplies the input image in which the shadow region detected using the difference image is specified to the hidden image generation unit 83 and the shadow image generation unit 85. Then, the processing is proceeds to step S47.

In step S47, as described with reference to FIG. 19, the shadow image generation unit 85 obtains (generates) a shadow image in which a shadow region to be added to the base image is specified, from among the input image specified by the shadow/shade region detection unit 82. Then, the processing proceeds to step S48.

In step S48, the storage unit 81 selects the shadow region extraction target image from the frame set, and supplies the selected image to the hidden image generation unit 83. Then, the processing proceeds to step S49.

In step S49, as described with reference to FIG. 19, the hidden image generation unit 83 uses the shadow region extraction target image from the storage unit 81 and input image in which the shadow region is specified from the shadow/shade region detection unit 82 so as to generate an image in which a portion that is a shadow region that is invisible hidden by the shadow in the base image but appears in the shadow region extraction target image is specified, as a hidden image.

The hidden image generation unit 83 supplies the hidden image to the shadow removing unit 84, and the processing proceeds from step S49 to step S50.

In step S50, the storage unit 81 selects a base image from the frame set and supplies the selected base image to the shadow removing unit 84. Then, the processing proceeds to step S51.

In step S51, as described in FIG. 20, the shadow removing unit 84 combines the hidden image from the hidden image generation unit 83 to the base image from the storage unit 81 so as to generate an image in which a portion that used to be invisible as a shadow region in the base image is now visible as a shadow removed image obtained by removing the shadow region from the base image.

Then, the shadow removing unit 84 supplies the shadow removed image to the combining unit 86, and the processing proceeds from step S51 to step S52.

In step S52, as described in FIG. 21, the shadow image generation unit 85 generates a new shadow image obtained by adding a new shadow (shadow region) to the shadow region of the shadow image, that is, generates a shadow image having an expanded shadow region in accordance with the virtual light source position from the virtual light source position setting unit 51.

Then, the shadow image generation unit 85 supplies the shadow image to the combining unit 86, and the processing proceeds from step S52 to step S53.

In step S53, the combining unit 86 combines the shadow removed image from the shadow removing unit 84 and the shadow image (shadow region of the shadow image) from the shadow image generation unit 85 so as to generate an output image obtained by adding a shadow that is an expanded shadow of the input image as an output image and outputs the generated image to the display apparatus 13, and then, the processing is finished.

Note that the combining unit 86 can output the shadow removed image and the shadow image as it is without combining them. In this case, the shadow removed image and the shadow image can be combined with each other at the time of display of the removed image and the shadow image similarly to the case described with reference to FIG. 5.

<Fourth Exemplary Configuration of Image Processing Apparatus 12>

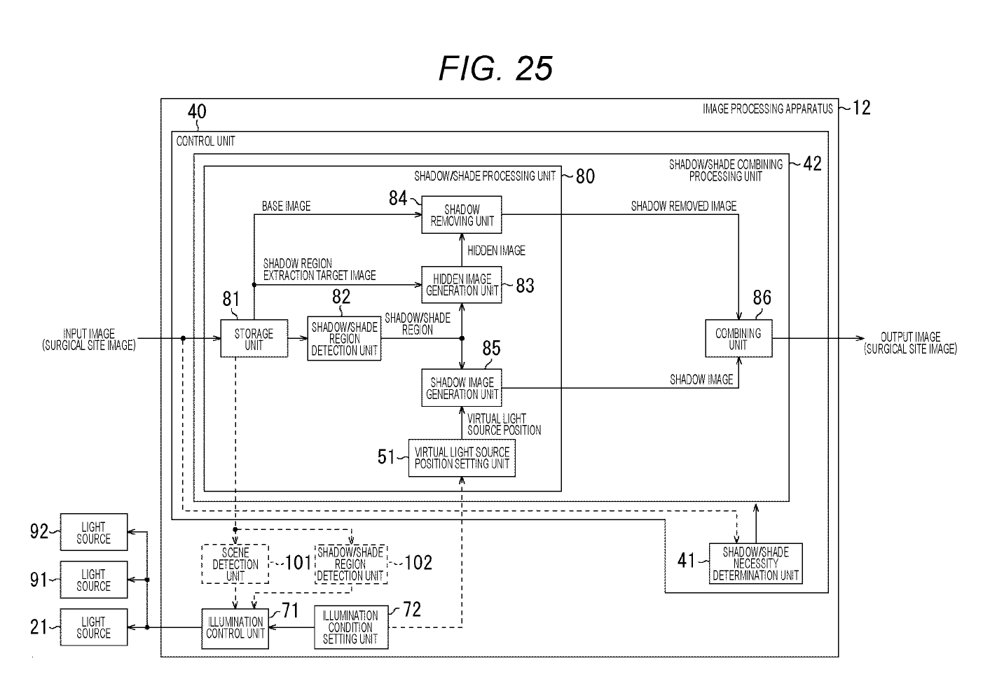

FIG. 25 is a block diagram illustrating a fourth exemplary configuration of the image processing apparatus 12 in FIG. 2.

Note that in the figure, portions corresponding to the case of FIG. 13 are denoted by the same reference numerals, and the description thereof will be omitted as appropriate below.

In FIG. 25, the image processing apparatus 12 includes the control unit 40, the illumination control unit 71, and the illumination condition setting unit 72.

The control unit 40 includes the shadow/shade necessity determination unit 41 and the shadow/shade combining processing unit 42.

The shadow/shade combining processing unit 42 includes the shadow/shade processing unit 80 and the combining unit 86.

The shadow/shade processing unit 80 includes the virtual light source position setting unit 51, the storage unit 81, the shadow/shade region detection unit 82, the hidden image generation unit 83, the shadow removing unit 84, and the shadow image generation unit 85.

Therefore, the image processing apparatus 12 of FIG. 25 is configured similarly to the case of FIG. 13.

Still, FIG. 25 differs from the case of FIG. 13 including the light source 21 alone as a light source and the control of the light source 21 alone is performed, in that the FIG. 25 also includes light sources 91 and 92 in addition to the light source 21, and that the illumination control unit 71 also controls light sources 91 and 92 in addition to the light source 21.

The light sources 91 and 92 can be provided on a trocar (not illustrated) as an piercing instrument attached to an abdominal wall or the like by opening a small hole for inserting the endoscope 11 (endoscope scope 32 of the endoscope 11 (FIG. 3)) and the treatment tool or the like into the body cavity, for example.

In addition, the light sources 91 and 92 can be added to the distal end of a treatment tool such as forceps to be inserted from the trocar, for example. In this case, the treatment tool to which the light sources 91 and 92 are added is held by the surgeon, the robot, or the like in a state inserted from the trocar.

In the image processing apparatus 12 of FIG. 25, the illumination control unit 71 controls not only the single light source 21 but also a plurality of light source, for example, three light sources 21, 91, and 92. With this control, it is possible to illuminate the subject under the illumination conditions with wider variations. As a result, it is possible to generate an output image to which a shadow (region) that has a stereoscopic effect has been added, for example.

Note that the image processing apparatus 12 of FIG. 25 may further include a scene detection unit 101 that detects a scene appearing in an input image and a shadow/shade region detection unit 102 that detects a shadow/shade (region) appearing in the input image.

In this case, the illumination control unit 71 can control the light source 21, 91, and 92 in accordance with the scene appearing in the input image detected by the shadow appearing in the input image detected by the scene detection unit 101 or the shadow/shade appearing in the input image detected by the shadow/shade region detection unit 102, separately from controlling the light sources 21, 91, and 92 in accordance with the (setting) illumination conditions supplied from the illumination condition setting unit 72.

That is, the illumination control unit 71 can control on/off of the light sources 21, 91, and 92, for example, that is, the position to illuminate the subject (position at which the illumination light for illuminating the subject is emitted) in accordance with the scene or shadow/shade appearing in the input image.

In addition, the illumination control unit 71 can control the intensity of the light source 21, 91, and 92, that is, the intensity of the illumination light illuminating the subject, for example, in accordance with the scene or shadow/shade appearing in the input image.

For example, in a case where a shadow of another subject appears on a surgical site appearing in a medical image as an input image, it is possible to selectively turn on the light source capable of emitting the illumination light from the direction that would not cause the shadow of the other subject to appear on the surgical site among the light source 21, 91, and 92, and possible to turn off the other light sources.

Note that whether the illumination control unit 71 controls the light sources 21, 91 and 92 in accordance with the illumination conditions supplied from the illumination condition setting unit 72, or in accordance with the scenes appearing in the input image detected by the scene detection unit 101 or shadow/shade appearing in the input image detected by the shadow/shade region detection unit 102 can be switched in accordance with user's operation, for example.

In addition, the illumination control unit 71 can control the light sources 21, 91, and 92 in accordance with user's operation.

For example, in a case where the user instructs a desired direction of adding a shadow, the illumination control unit 71 can selectively turn on the light source alone capable of emitting illumination light from a position that generates a shadow in the direction instructed by the user among the light sources 21, 91, and 92 and can turn off the other light sources.

In addition, for example, in a case where the user instructs the density of the shadow, the illumination control unit 71 controls the intensity of the light source to be needed among the light sources 21, 91, and 92 to the intensity corresponding to the density of the shadow instructed by the user.

Note that in FIG. 25, the shadow/shade region detection unit 102 can be substituted by the shadow/shade region detection unit 82.

<Fifth Exemplary Configuration of Image Processing Apparatus 12>

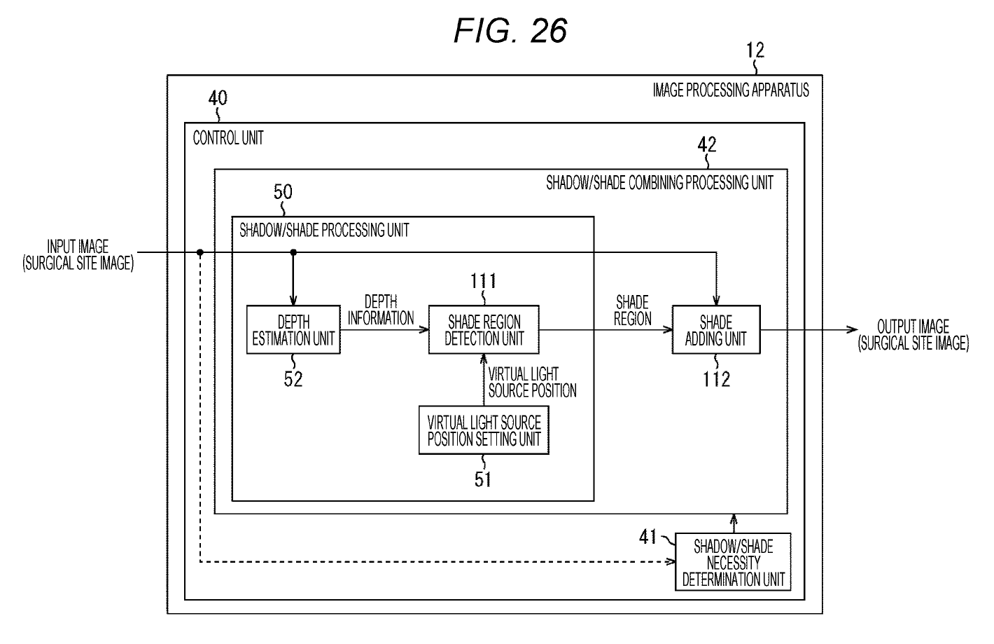

FIG. 26 is a block diagram illustrating a fifth exemplary configuration of the image processing apparatus 12 in FIG. 2.

Note that in the figure, portions corresponding to the case of FIG. 5 are denoted by the same reference numerals, and the description thereof will be omitted as appropriate below.

In FIG. 26, the image processing apparatus 12 includes the control unit 40.

The control unit 40 includes the shadow/shade necessity determination unit 41 and the shadow/shade combining processing unit 42.

The shadow/shade combining processing unit 42 includes the shadow/shade processing unit 50 and a shade adding unit 112.

The shadow/shade processing unit 50 includes the virtual light source position setting unit 51, the depth estimation unit 52, and a shade region detection unit 111.

Therefore, the image processing apparatus 12 in FIG. 26 is similar to the case of FIG. 5 in that it includes the control unit 40, and is similar to the case of FIG. 5 in that the control unit 40 includes the shadow/shade necessity determination unit 41 and the shadow/shade combining processing unit 42, in that the shadow/shade combining processing unit 42 includes the shadow/shade processing unit 50, and in that the shadow/shade processing unit 50 includes the virtual light source position setting unit 51 and the depth estimation unit 52.

However, the image processing apparatus 12 of FIG. 26 differs from the case of FIG. 5 in that the shadow/shade combining processing unit 42 includes the shade adding unit 112 instead of the combining unit 54, and in that the shadow/shade processing unit 50 includes the shade region detection unit 111 instead of the shadow image generation unit 53.

The shade region detection unit 111 receives the virtual light source position supplied from the virtual light source position setting unit 51 and receives the depth information supplied from the depth estimation unit 52.

The shade region detection unit 111 detects a shade region of the shade generated by the virtual light source on the basis of the virtual light source position from the virtual light source position setting unit 51 and the depth information from the depth estimation unit 52, and supplies the detected region to the shade adding unit 112.

The shade adding unit 112 receives the shade region supplied from the shade region detection unit 111, and receives a medical image as an input image supplied from the camera 22.

The shade adding unit 112, for example, combines the shade region from the shade region detection unit 111 to the input image from the camera 22 to generate an output image obtained by adding the shade region to the input image, and outputs the generated image to the display apparatus 13.

Meanwhile, combining of the input image and the shadow region in the shade adding unit 112 can adopt alpha blending similar to the case of the combining unit 54 of FIG. 5, for example.

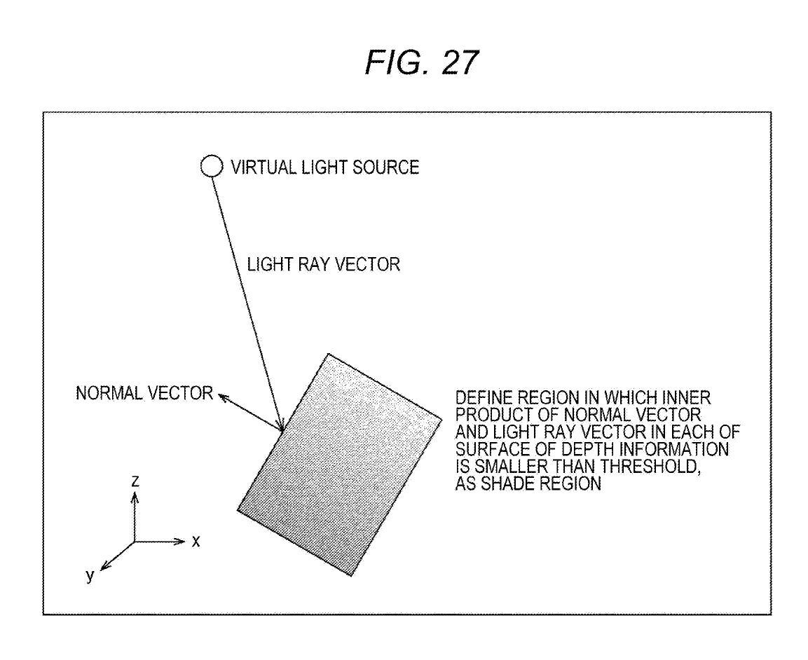

FIG. 27 is a diagram illustrating an exemplary shade region detection by the shade region detection unit 111 of FIG. 26.

In a three-dimensional space (hereinafter also referred to as a depth space) defined by an xy plane representing the position of each of pixels of an input image and a z-axis representing depth information of a subject appearing in each of pixels, a vector representing a light ray directed from the virtual light source position toward a point as the depth information is defined as a light ray vector.

Regarding the depth information of each of pixels, the shade region detection unit 111 obtains an inner product of a normal vector representing the normal direction at the point as the depth information and a light ray vector directed to the point as the depth information.

Then, the shade region detection unit 111 detects, as a shade region, a region including pixels of depth information in which the size of the inner product is equal to or less than (or less than) the predetermined threshold.

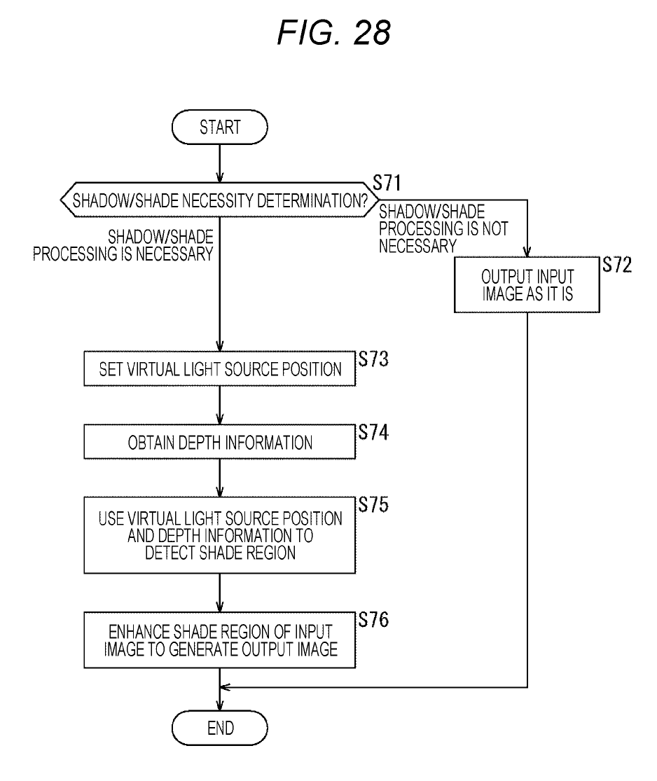

FIG. 28 is a flowchart illustrating exemplary processing of the image processing apparatus 12 in FIG. 26.

In steps S71 to S74, processing similar to steps S11 to S14 of FIG. 9 is to be respectively performed.

Then, in step S75, using the virtual light source position from the virtual light source position setting unit 51 and the depth information from the depth estimation unit 52, the shade region detection unit 111 detects a shadow region of the shade generated by the virtual light source as described in FIG. 27.

The shade region detection unit 111 supplies the shadow region to the shade adding unit 112, and the processing proceeds from step S75 to step S76.

In step S76, the shade adding unit 112 combines the shade region from the shade region detection unit 111 to the input image from the camera 22, to generate an output image obtained by adding a shade region to the input image, that is, generate an output image in which the shade of the input image is emphasized, and outputs the generated output image to the display apparatus 13, and then, the processing is finished.

With the image processing apparatus 12 of FIG. 26, shades are emphasized by addition of a shade region to the input image, enabling the user to feel irregularity effects and stereoscopic effect more easily.

<Description of Computer According to Present Technology>

The above-described series of processing of the image processing apparatus 12 can be executed in hardware or with software. In a case where the series of processes is executed with software, a program included in the software is installed in a general-purpose computer, or the like.

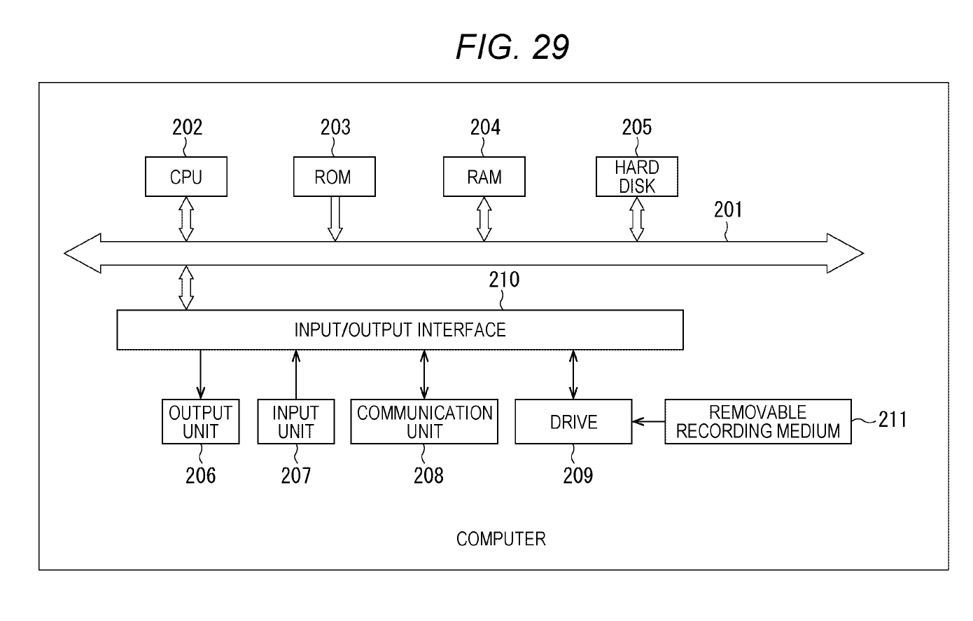

FIG. 29 is a block diagram illustrating an exemplary configuration of a computer according to an embodiment, in which a program that executes the above-described series of processes is installed.

The program can be previously recorded in a hard disk 205 or a ROM 203, as a recording medium built into the computer.

Alternatively, the program can be stored (recorded) in a removable recording medium 211. The removable recording medium 211 can be supplied as package software. Examples of the removable recording medium 211 include a flexible disk, a compact disc read only memory (CD-ROM), a magneto optical (MO) disk, a digital versatile disc (DVD), a magnetic disk, a semiconductor memory, or the like.

Note that the program can be installed from the above-described removable recording medium 211 to the computer. Alternatively, the program can be downloaded to the computer via a communication network or a broadcasting network, and can be installed onto the built-in hard disk 205. Specifically, the program can be transferred, for example, from a downloading site to the computer wirelessly via an artificial satellite for digital satellite broadcasting, or can be transferred by wire to the computer via a network such as a local area network (LAN) and the Internet.

The computer incorporates a central processing unit (CPU) 202. The CPU 202 is connected to an input/output interface 210 via a bus 201.

When an instruction is input into the CPU 202 by operation, or the like, by a user, on an input unit 207 via the input/output interface 210, the CPU 202 executes a program stored in the read only memory (ROM) 203 according to the instruction. Alternatively, the CPU 202 loads the program stored in the hard disk 205 to a random access memory (RAM) 204 and executes the program.

With this procedure, the CPU 202 executes processing according to the above-described flowchart or processing done by the above-described configuration in the block diagram. Subsequently, the CPU 202 permits a processing result, as required, for example, to be output from an output unit 206, transmitted from a communication unit 208, or recorded in the hard disk 205, via the input/output interface 210.

Note that the input unit 207 includes a keyboard, a mouse, a microphone, and the like. In addition, the output unit 206 includes a liquid crystal display (LCD), a speaker, and the like.

In this description, processing executed by a computer in accordance with a program need not be performed in time series along the order described in the flowchart. That is, processing executed by the computer according to the program includes processing executed in Parallel or separately (e.g. parallel processing, or object processing).

In addition, the program can be processed by one computer (processor) or can be processed with distributed processing by a plurality of computers. Furthermore, the program can be transferred to a remote computer and be executed.

Furthermore, in the present description, the system represents a set of multiple constituents (devices, modules (parts), or the like). In other words, all the constituents may be in a same housing but they do not have to be in the same housing. Accordingly, a plurality of apparatuses, housed in separate housings, connected via a network can be a system. An apparatus in which a plurality of modules is housed in one housing can also be a system.

Note that embodiments of the present technology are not limited to the above-described embodiments but can be modified in a variety of ways within a scope of the present technology.

Moreover, the present technology can also be applied to a medical device having a function of photographing a medical image in which a surgical site of a living body or the like appears, such as a medical electron microscope (surgical microscope) in addition to the medical endoscope system. Furthermore, the present technology can be applied to devices having a function of photographing arbitrary images in addition to medical images.

In addition, the present technology can be configured as a form of cloud computing in which one function is shared in cooperation for processing among a plurality of apparatuses via a network.

Moreover, each of steps described in the above flowcharts can be executed on one apparatus or shared by a plurality of apparatuses for processing.

Furthermore, in a case where one step includes a plurality of stages of processing, the plurality of stages of processing included in the one step can be executed on one apparatus or can be shared by a plurality of apparatuses.

In addition, effects described herein are provided for purposes of exemplary illustration and are not intended to be limiting. Still other effects may also be contemplated.

Note that the present technology can be configured as follows.

<1>

An image processing apparatus including a control unit that determines whether to add or suppress shadow/shade to a medical image and controls to generate a shadow/shade corrected image on the basis of a result of the determination.

<2>

The image processing apparatus according to <1>,

in which the control unit performs the determination in accordance with an input from a user.

<3>

The image processing apparatus according to <1> or <2>,

in which the control unit performs the determination in accordance with the medical image.

<4>

The image processing apparatus according to any of <1> to <3>,

in which the control unit performs the determination in accordance with a use situation of a treatment tool.

<5>

The image processing apparatus according to any of <1> to <4>,

in which the control unit controls to generate the shadow/shade corrected image of a shadow occurring in a specific subject of the medical image by a virtual light source.

<6>

The image processing apparatus according to <5>,

in which the control unit estimates a depth of the subject, and controls to generate the shadow/shade corrected image on the basis of the depth.

<7>

The image processing apparatus according to <6>,

in which the control unit controls to enable a distance between a light source position of an imaging unit that photographs the medical image and a position of the virtual light source to be a predetermined distance or below.

<8>

The image processing apparatus according to <6>,

in which the control unit controls not to generate the shadow/shade corrected image for the subject in a case where a distance in a depth direction between the subject and a shadow region generated by the virtual light source is a predetermined distance or more.

<9>

The image processing apparatus according to any of <6> to <8>,

in which the medical image is formed with two images having parallax, and

the depth is estimated from parallax information of the subject of the two images.

<10>

The image processing apparatus according to any of <1> to <9>,

in which the control unit further specifies a target object from the medical image, and

controls to generate the shadow/shade corrected image as the target object as a target.

<11>

The image processing apparatus according to <10>, further including

an object setting unit that sets the target object.

<12>

The image processing apparatus according to <10>or <11>,

in which the control unit controls to generate the shadow/shade corrected image by setting a thickness of the target object to a predetermined thickness.

<13>

The image processing apparatus according to any of <1> to <4>,

in which the control unit controls to generate the shadow/shade corrected image using a plurality of the medical images obtained by photographing the subject with mutually different illumination conditions.

<14>

The image processing apparatus according to <13>, further including

an illumination condition setting unit that sets the illumination conditions.

<15>

The image processing apparatus according to any of <1> to <14>,

in which the control unit generates a shadow image in which a shadow appears as the shadow/shade corrected image.

<16>

The image processing apparatus according to any of <1> to <14>,

in which the control unit generates an output image in which a shadow has been added to the medical image by combining a shadow image in which a shadow appears and the medical image, as the shadow/shade corrected image.

<17>

The image processing apparatus according to <5>,

in which a position at which a longitudinal direction of a predetermined subject appearing in the medical image does not overlap with an optical axis of the virtual light source is set as a position of the virtual light source.

<18>

An image processing method including steps of:

determining whether to add or suppress shadow/shade to a medical image; and

controlling to generate a shadow/shade corrected image on the basis of a result of the determination.

<19>

A program that causes a computer to function as a control unit that determines whether to add or suppress shadow/shade to a medical image, and

controls to generate a shadow/shade corrected image on the basis of a result of the determination.

<20>

A surgical system including:

an endoscope that photographs a medical image;

a light source that emits illumination light for illuminating a subject; and

an image processing apparatus that performs image processing on the medical image of the subject illuminated by the illumination light, obtained by photographing with the endoscope,

in which the image processing apparatus includes a control unit that determines whether to add or suppress shadow/shade to a medical image, and

controls to generate a shadow/shade corrected image on the basis of a result of the determination.

<O1>

An image processing apparatus including:

a determination unit that performs shadow/shade necessity determination of whether to perform shadow/shade processing of adding or suppressing shadow/shade on a medical image in which a surgical site appears; and

a shadow/shade processing unit that performs the shadow/shade processing on the medical image in accordance with a determination result of the shadow/shade necessity determination.

<O2>

The image processing apparatus according to <O1>,

in which the determination unit performs the shadow/shade necessity determination in accordance with an input from a user.

<O3>

The image processing apparatus according to <O1> or <o2>,

in which the determination unit performs the shadow/shade necessity determination in accordance with the medical image.

<O4>

The image processing apparatus according to any of <O1> to <O3>,

in which the determination unit performs the shadow/shade necessity determination in accordance with a use situation of a treatment tool.

<O5>

The image processing apparatus according to any of <O1> to <O4>, further including:

a depth estimation unit that estimates a depth of a subject appearing in each of pixels of the medical image;

a virtual light source position setting unit that sets a virtual light source position of a virtual light source, and

a shadow image generation unit that generates a shadow image of a shadow generated by the virtual light source on the basis of the depth of the subject and the virtual light source position,

in which the shadow/shade processing unit combines the medical image and the shadow image to generate an output image in which a shadow is added to the medical image.

<O6>

The image processing apparatus according to <O5>,

in which the virtual light source position setting unit limits a distance between the optical axis of a camera that photographs the medical image and the virtual light source position within a predetermined distance.

<O7>

The image processing apparatus according to <O5> or <O6>,

in which the shadow/shade processing unit limits addition of shadows for a subject in which a distance in the depth direction between a subject appearing in the medical image and a shadow generated by the virtual light source for the subject is a predetermined distance or more.

<O8>

The image processing apparatus according to any of <O5> to <O7>,

in which the virtual light source position setting unit sets a position at which a longitudinal direction of a predetermined subject appearing in the medical image does not overlap with an optical axis of the virtual light source as the virtual light source position.

<O9>

The image processing apparatus according to any of <O5> to <O8>,

in which the medical image is a three-dimensional (3D) image, and

the depth estimation unit estimates a depth of a subject appearing in each of pixels of the medical image on the basis of the 3D image.

<O10>

The image processing apparatus according to any of <O1> to <O9> further including an object detection unit that detects a target object to be a target of the shadow/shade processing from the medical image,

in which the shadow/shade processing unit performs the shadow/shade processing on the target object as a target.

<O11>

The image processing apparatus according to <O10>, further including

an object setting unit that sets the target object.

<O12>

The image processing apparatus according to <O10> or <O11>,

in which the shadow/shade processing unit performs the shadow/shade processing in consideration of a predetermined thickness as a thickness of the target object.

<O13>

The image processing apparatus according to any of <O1> to <O4>, further including:

a shadow/shade region detection unit that uses a plurality of frames photographed with mutually different illumination conditions among the frames of the medical image photographed with different illumination conditions for illuminating a subject appearing in the medical image so as to detect a shadow/shade region having shadow/shade in the medical image; and

a virtual light source position setting unit that sets a virtual light source position of a virtual light source,

in which the shadow/shade processing unit performs the shadow/shade processing in the shadow/shade region in accordance with the virtual light source position.

<O14>

The image processing apparatus according to <O13>,

in which the shadow/shade processing unit

generates a shadow removed image in which a shadow region having a shadow has been removed in a base image when a latest frame among the plurality of frames is defined as the base image,

generates a shadow image in which a new shadow has been added to a shadow region having a shadow in a shadow region extraction target image using the shadow region extraction target image and the virtual light source position when one frame of the plurality of frames is defined as the shadow region extraction target image, and

generates an output image in which the new shadow has been added to the medical image by combining the shadow removed image and the shadow image.

<O15>

The image processing apparatus according to any of <O1> to <O4> further including:

a depth estimation unit that estimates a depth of a subject appearing in each of pixels of the medical image;

a virtual light source position setting unit that sets a virtual light source position of a virtual light source; and

a shade region detection unit that detects a shade region of a shade generated by the virtual light source on the basis of the depth of the subject and the virtual light source position,

in which the shadow/shade processing unit generates an output image in which the shade region has been added to the medical image.

<O16>

An image processing method including:

performing shadow/shade necessity determination of whether to perform shadow/shade processing of adding or suppressing shadow/shade on a medical image in which a surgical site appears; and

performing the shadow/shade processing on the medical image in accordance with a determination result of the shadow/shade necessity determination.

<O17>

A program that causes a computer to function as:

a determination unit that performs shadow/shade necessity determination of whether to perform shadow/shade processing of adding or suppressing shadow/shade on a medical image in which a surgical site appears; and

a shadow/shade processing unit that performs the shadow/shade processing on the medical image in accordance with a determination result of the shadow/shade necessity determination.

<O18>

A surgical system including:

an endoscope that photographs an image;

a light source that emits illumination light for illuminating a subject; and

an image processing unit that performs image processing of a medical image in which a surgical site appears obtained with the endoscope by photographing the surgical site illuminated by the illumination light,

in which the image processing apparatus includes:

a determination unit that perform shadow/shade necessity determination of whether to perform shadow/shade processing of adding or suppressing shadow/shade onto a medical image in which a surgical site appears; and

a shadow/shade processing unit that performs the shadow/shade processing on the medical image in accordance with a determination result of the shadow/shade necessity determination.

### REFERENCE SIGNS LIST

11 Endoscope
12 Image processing apparatus
13 Display apparatus
21 Light source
22 Camera
31 Camera head
32 Endoscope scope
33 Forceps
40 Control unit
41 Shadow/shade necessity determination unit
42 Shadow/shade combining processing unit
50 Shadow/shade processing unit
51 Virtual light source position setting unit
52 Depth estimation unit
53 Shadow image generation unit
54 Combining unit
61 Object setting unit
62 Object detection unit
71 Illumination control unit
72 Illumination condition setting unit
80 Shadow/shade processing unit
81 Storage unit
82 Shadow/shade region detection unit
83 Hidden Image Generation unit
84 Shade removing unit
85 Shadow image generation unit
86 Combining unit
91, 92 Light source
101 Scene detection unit
102 Shadow/shade region detection unit
111 Shade region detection unit
112 Shade adding unit
201 Bus
202 CPU
203 ROM
204 RAM
205 Hard disk
206 Output unit
207 Input unit
208 Communication unit
209 Drive
210 Input/output interface
211 Removable recording medium

1. An image processing apparatus comprising a control unit that determines whether to add or suppress shadow/shade to a medical image and controls to generate a shadow/shade corrected image on the basis of a result of the determination.

2. The image processing apparatus according to claim 1,
wherein the control unit performs the determination in accordance with an input from a user.

3. The image processing apparatus according to claim 1,
wherein the control unit performs the determination in accordance with the medical image.

4. The image processing apparatus according to claim 1,
wherein the control unit performs the determination in accordance with a use situation of a treatment tool.

5. The image processing apparatus according to claim 1,
wherein the control unit controls to generate the shadow/shade corrected image of a shadow occurring in a specific subject of the medical image by a virtual light source.

6. The image processing apparatus according to claim
wherein the control unit estimates a depth of the subject, and controls to generate the shadow/shade corrected image on the basis of the depth.

7. The image processing apparatus according to claim 6,
wherein the control unit controls to enable a distance between a light source position of an imaging unit that photographs the medical image and a position of the virtual light source to be a predetermined distance or below.

8. The image processing apparatus according to claim 6,
wherein the control unit controls not to generate the shadow/shade corrected image for the subject in a case where a distance in a depth direction between the subject and a shadow region generated by the virtual light source is a predetermined distance or more.

9. The image processing apparatus according to claim 6,
wherein the medical image is formed with two images having parallax, and
the depth is estimated from parallax information of the subject of the two images.

10. The image processing apparatus according to claim 1,
wherein the control unit further specifies a target object from the medical image, and
controls to generate the shadow/shade corrected image as the target object as a target.

11. The image processing apparatus according to claim 10, further comprising
an object setting unit that sets the target object.

12. The image processing apparatus according to claim 10,
wherein the control unit controls to generate the shadow/shade corrected image by setting a thickness of the target object to a predetermined thickness.

13. The image processing apparatus according to claim 1,
wherein the control unit controls to generate the shadow/shade corrected image using a plurality of the medical images obtained by photographing the subject with mutually different illumination conditions.

14. The image processing apparatus according to claim 13, further comprising
an illumination condition setting unit that sets the illumination conditions.

15. The image processing apparatus according to claim 1,
wherein the control unit generates a shadow image in which a shadow appears as the shadow/shade corrected image.

16. The image processing apparatus according to claim 1,
wherein the control unit generates an output image in which a shadow has been added to the medical image by combining a shadow image in which a shadow appears and the medical image, as the shadow/shade corrected image.

17. The image processing apparatus according to claim 5,
wherein a position at which a longitudinal direction of a predetermined subject appearing in the medical image does not overlap with an optical axis of the virtual light source is set as a position of the virtual light source.

18. An image processing method comprising steps of:
determining whether to add or suppress shadow/shade to a medical image; and
controlling to generate a shadow/shade corrected image on the basis of a result of the determination.

19. A program that causes a computer to function as a control unit that determines whether to add or suppress shadow/shade to a medical image, and
controls to generate a shadow/shade corrected image on the basis of a result of the determination.

20. A surgical system comprising:
an endoscope that photographs a medical image;
a light source that emits illumination light for illuminating a subject; and
an image processing apparatus that performs image processing on the medical image of the subject illuminated by the illumination light obtained by photographing with the endoscope,
wherein the image processing apparatus includes a control unit that
determines whether to add or suppress shadow/shade to a medical image, and
controls to generate a shadow/shade corrected image on the basis of a result of the determination.
